(19) United States
(12) Patent Application Publication
(10) Pub. No.: US 2009/0110674 A1
(43) Pub. Date: Apr. 30, 2009

(54) Health supplement

(76) Inventor: Nicos C. Loizou, Virginia Beach, VA (US)

Correspondence Address: WILLIAMS G. SYKES, 3669 SEAGULL BLUFF DRIVE, VIRGINIA BEACH, VA, 23455, US

(21) Appl. No.: 12/288,679

(22) Filed: Oct. 22, 2008

Related U.S. Application Data
(60) Provisional application No. 61/000,121, filed on Oct. 24, 2007.

(51) Int. Cl. A61K 38/54

(52) U.S. Cl. 424/94.2

(57) ABSTRACT

A health supplement consisting of numerous ingredients from several general groups including: anti-aging, anti-oxidant, vitamins, minerals, and elemental substances including metals. The supplement is specifically formulated for maximizing synergistic interaction of the ingredients while eliminating harmful interaction among the ingredients. A holistic approach to health is followed with ingredients to aid all systems of the human body. By addressing all human body systems, the supplement acts as a precursor to anti-aging and, when body systems are detoxified and oxidation is controlled, the effect of aging may be reversed.

### RELATED APPLICATIONS

This application is based in part on, and claims priority to, U.S. Provisional Patent Application Ser. No. 61/000,121 titled Forever Young Youth Formula, filed Oct. 24, 2007, which is included herein in its entirety by reference.

### FIELD OF THE INVENTION

The invention pertains to health and nutritional supplements and, more particularly, to a health supplement provided in capsular or liquid form.

### BACKGROUND OF THE INVENTION

Health supplements are used by millions of Americans everyday. These supplements range from single-ingredient vitamin supplements to multi-vitamins to prescription supplements incorporating hormone therapy and other medicinal treatments. A broad number of these health supplements are readily available to the public in over-the-counter formulations, many touting condition specific uses, such as the recent spate of “ultimate anti-aging formulations.” One of these formulations contains 65 components and suggests ingestion of over 10,000 mg of the combination per day that, due to limitations of tablet size, requires ingesting ten tablets each day.

More popular brands of multivitamins contain between 26 ingredients (Theragran-M—by Mead Johnson) and 53 ingredients (Maxilife Phyto—by Twinlab). These “shotgun” approaches of incorporating all possible useful ingredients face not only serious criticisms, but also present serious potential dangers to a user. For example, riboflavin (vitamin B₂) is known to be toxic in doses above 10 mg per day. Many supplement formulations exceed this limit, posing a potential health risk to the users.

Many health supplement formulations include substantial numbers of herbal compounds. Herbal compounds naturally contain numerous active chemical groups. These active chemical groups compete for overall effect. For example, lemon fruit has an acid content, but counterintuitively, has an alkaline effect within the human body. Grapefruit contains antioxidant herbal substances that can interfere with certain drugs. It is intuitive that indiscriminate combinations of numerous herbs, each containing numerous key constituents, might have a subtractive overall effect. Garlic is often used in formulations for its antioxidant properties, but may have the side effect of combining with other components in the formulation to, for example, exceed safe limits of selenium (200 mcg/day), due to garlic's relatively high selenium content. Therefore, many supplements on the market incorporating herbal compounds may be more harmful than “healthful.”

One promising way to incorporate important supplement components while reducing the potential for high dose toxicity is to utilize synergistic relationships among components. “Pharmacologic synergy” is a complementary, superadditive response resulting from the combination of two or more agents. For example, there are numerous examples of synergy between antioxidant vitamins. U.S. Pat. No. 5,994,322 to Masuda cites a lecithin-vitamin B₁₂ combination as synergistic. The oxidative synergy of vitamin E and phospholipids have been demonstrated as better than vitamin E alone. Vitamin A and vitamin E have more antioxidant synergy with phosphatidylcholine liposomes than the respective single components. Other studies on synergy between numerous compounds for a focused outcome are surprisingly sparse.

Conversely, many supplement formulations do not provide enough of certain supplements.

One of the more important focuses of health supplements is the reduction of free radicals. Free radicals are associated with aging of the brain. Oxidative injury to the nervous system has been documented in diseases such as AIDS-associated dementia, Alzheimer's disease, benign senile forgetfulness (pre-Alzheimer's disorder), Down's syndrome-associated dementia, Lewy body dementia, multi-infarct dementia, multiple sclerosis, Parkinson's disease-associated dementia, tardive dyskinesia, Wernicke-Korsakoff syndrome, and alcoholism-associated dementia. Indeed, oxidative injury may be the final common pathway leading to cell death. Numerous studies have shown benefit from the use of antioxidants in many of these disorders.

Antioxidants are substances that protect against oxidative stress damage caused by free radicals. There are four generally recognized groups of antioxidants: plant extracts, vitamins, amino acids, and minerals. Vitamins are organic substances provided in relatively small quantities from the environment that are necessary for the maintenance of health. Precursors (e.g., carotene for vitamin A), vitamins having multiple forms (e.g., pyridoxine, pyridoxal, pyridoxamine), and essential oligopeptides are typically referred to as “vitamers,” a term used synonymously with the term “vitamin.”

As discussed above, prior art supplement compositions generally take a “shotgun” approach of providing supplements instead of providing specific formulations for other than a generalized “anti-aging” or “energy” formulation. However, U.S. Pat. No. 6,048,846 to Cochran discloses a combination of supplement components designed to fight the causes of disease. The essential combination disclosed is use of at least one hormone with the supplement components. However, hormone administration can be dangerous given the potential downstream effects.

The present invention overcomes the problems associated with a “shotgun” supplement approach. The dosage ranges and use of synergistic component relationships overcome problems with potential toxicity.

### SUMMARY OF THE INVENTION

The present invention provides a health supplement consisting of numerous ingredients from several general groups: anti-aging, anti-oxidant, vitamins, minerals, and elemental substances including metals. The supplement is specifically formulated for maximizing synergistic interaction of the ingredients while eliminating harmful interaction among the ingredients. A holistic approach to health is followed with ingredients to aid all systems of the human body. By addressing all human body systems, the supplement acts as a precursor to anti-aging, and when body systems are detoxified and oxidation is controlled, the effect of aging may be reversed.

It is, therefore, an object of the invention to provide an anti-aging health supplement.

It is another object of the invention to provide an anti-aging health supplement comprising all natural ingredients.

It is an additional object of the invention to provide an anti-aging health supplement comprising vitamins, enzymes, amino acids, herbs, and a full spectrum of minerals and trace elements.

It is a further object of the invention to provide an anti-aging health supplement in both a powder (i.e., capsular) and a liquid form.

It is a still further object of the invention to provide an anti-aging health supplement that may be delivered in physically manageable doses.

It is yet another object of the invention to provide an anti-aging health supplement that avoids any quantity of a constituent that is potentially harmful to the human body.

### DETAILED DESCRIPTION OF THE PREFERRED EMBODIMENT

The present invention provides a health supplement consisting of numerous ingredients from several general groups: anti-aging, anti-oxidant, vitamins, minerals, and elemental substances including metals.

Many theories on aging exist today as there are over 5000 known researchers in the field worldwide, each with a common goal: defeating and/or reversing the human aging process. The present inventor began research in 1983, and with the help of today's available data and information, has come to the conclusion that developing an effective anti-aging formula is possible. Such a formula could not only re-energize the body but could conceivably stop and even reverses the whole aging process.

The formula of the present invention is believed to re-energize the body and stop and even reverses the aging process. The formula consists of all natural ingredients and, based on extensive research, is believed to have no general side affects based on either individual constituents or on interactions therebetween.

The novel formula combines different groups of ingredients, for example: Vitamins, Enzymes, Amino Acids, Herbs, and a full spectrum of minerals and trace elements. The resulting combination of these ingredients results in a composition that appears to stop the aging process and possibly reverses the aging process. All ingredients work together synergistically, and have been chosen to eliminate negative interactions among the ingredients. In other words, the formula is engineered to ensure 100% “compatibility” among the ingredients.

The formula of the present invention contributes to other significant results, which are beneficial to the human body, and are necessary to begin reversing aging. Some of the abilities of the novel formula appear to include at least:
improving and curing digestive problems;
lowering LDL (bad cholesterol);
treating diabetes and arthritis;
promoting longevity by being a powerful anti-oxidant;
improving poor blood circulation;
providing strong anti-cancer and anti-AIDS agents;
facilitating regeneration and reproduction of cells starting from the conceiving stage;
treating heart problems, liver cirrhosis and other liver problems caused by liver damage from toxic substances;
providing a renewing effect on the kidneys;
boosting the immune system by providing advanced immune protection against diseases;
detoxifying the blood;
relieving the symptoms of Alzheimer's disease;
ensuring a healthy nervous system by providing a micro-formula within a formula;
promoting hormone balance;
improving memory;
restoring graying hair to its natural color and preventing hair loss;
supporting vision health;
providing joint support
providing cardiovascular support;
promoting brain function by maintaining healthy neurons and neurotransmitters allowing a person to stay focused and have a better comprehension level; and
contributing to weight loss.

The benefits of the novel formulation go far beyond these major points. The formula may be used as a necessary blood nutrient. One of the ingredients of the formula is a natural substance called DHEA which is produced in the adrenal gland and brain and is synthesized in the adrenal cortex. As humans age, DHEA levels diminish and, consequently, need to be supplemented. Some studies show that such supplementation boosts the activity of natural killer cells and also helps prevent depression.

One of the functions of the novel formula is that energy levels are improved as the formula enhances the process of breaking down food into proteins. This vital role in protein synthesis stimulates pituitary release of growth hormones.

The formulae is also a liver cleanser and enhancer, and helps to defend against toxicity in the body. The formula also helps regulate thyroid hormone metabolism.

In summary, according to the inventor's studies and research, the major factor causing aging is OXIDATION! In other words, the air, the water, and the food breathed and ingested are major contributors to aging. However, each is a necessity to the human body—human life is not possible without them. The dilemma is resolved if the body can be deoxidized at a rate faster than it is being oxidized. When this occurs, it appears that the aging process may be reduced. For example, if the body oxidizes 10% daily and a 10% deoxidization can be sustained, aging may be stopped. If, on the other hand, the body oxidizes 10% daily but a 15% deoxidization occurs, aging should be reversed.

Human cells reproduce in cycles, and if every new cycle of cells comes out less oxidized than the previous generation of cells, a point is reached where the body will be completely deoxidized, thereby reversing the aging process.

The two factors that generally unknown are: first, most humans don't know how much oxidization is occurring in their bodies; and second, most humans don't know how to manage the deoxidization of their bodies to deoxidize at a faster rate than oxidation without causing harm.

The formula of the invention shows great potential for promoting health and harmony among all vital organs, factors necessary for reversing the aging process and staying young.

The formula is embodied in a health supplement that may be provided in a powder/granular form, typically packaged in capsules as well as a liquid form or as an additive to a liquid such as water. It will be recognized that other delivery forms for the novel supplement may be utilized to meet a particular operating circumstance or environment. Consequently, the invention is not considered limited to a particular delivery system but covers any and all suitable delivery systems.

The specific constituents are shown in Table I for both capsular and liquid forms.

TABLE I

| | | Original | | Powder | | | |
|---|---|---|---|---|---|---|---|
| | | Formula | | (Capsule) | | Liquid | |
| Acacia leaf extract | | | | 5 | mg | | |
| Acidophilus | Medicago sativa | 2.0 | mg | | | | |
| Alfalfa | | 35 | mg | 35 | mg | | |
| Alpha Lipoic Acid | | 7.0 | mg | 10 | mg | 50 | mcg |
| Amylase | (from pure plant enzymes) | 36 | USP units | 5.0 | mg | 2.5 | mg |
| Astragalus membranaceus | (syn. Astragalus propinquus) | 36.56 | mg | 75 | mg | 2.5 | mg |
| Biotin | | 120 | mcg | 1000 | mcg | 1000 | mcg |
| Bismuth | | 2.5 | mcg | | | | |
| Black Cherry | | 2.0 | mg | | | | |
| Bromelain | (from pure plant enzymes) | 1.0 | mg | 5.0 | mg | | |
| Calcium | | 6.7 | mg | 10 | mg | 2.5 | mg |
| Carnosine | | | | 5.0 | mg | 3.5 | mg |
| Cellulase | (from pure plant enzymes) | 1.0 | mg | 5.0 | mg | 2.5 | mg |
| Chlorella | | 32 | mg | 5.0 | mg | | |
| Chondroitin sulfate | | 13 | mg | 10 | mg | | |
| Chromium Picolinate | | 2.5 | mcg | 5.0 | mcg | | |
| Chrysin | | | | 15 | mg | | |
| Clintonia borealis | Lily extract | | | 5.0 | mg | | |
| Copper | | 2 | mcg | 3.5 | mcg | | |
| Co-Q10 | | 25 | mg | 15 | mg | 5.0 | mg |
| Cyanotis Vaga | | | | 20 | mg | 2.5 | mg |
| Cystoseria canariensis | (from Brown Sea Algae) | | | 7.5 | mg | | |
| DHEA | | 3.0 | mg | 3.0 | mg | | |
| Emblica officinalis | | | | 25 | mg | 2.0 | mg |
| Ester-C | | 25 | mg | 10 | mg | | |
| Fucoxanthin | (from Brown Seaweed) | | | 10 | mg | | |
| Ganoderma lucidum | Ling zhi red Reishi Mushroom | | | 65 | mg | 10 | mg |
| Gingo Biloba | | | | | | 1.5 | mg |
| Ginseng (Panax) | | | | 5 | mg | 7.5 | mg |
| Glucosamin | | 13 | mg | 10 | mg | | |
| Glutamic Acid HCL | | 2.25 | mg | | | | |
| Glycine | | | | 3.5 | mg | | |
| Gold | | 2.0 | mcg | 1.5 | mcg | | |
| Grape seed extract | | 28.89 | mg | 85 | mg | 7.5 | mg |
| Guarana Seed Extract | | | | | | 7.5 | mg |
| Hemicellulase | | 0.11 | mg | | | | |
| Horse chestnut seed extract | | | | 5 | mg | | |
| Iodine | | 2.5 | mcg | | | | |
| Lactase | (from pure plant enzymes) | | | 5 | mg | 2.5 | mg |
| Lactobacillus, Bulgarius, | | 1.5 | mg | 2.5 | mg | | |
| Bifidus | | | | | | | |
| L-Arginine | | 45 | mg | 10 | mg | 1.5 | mg |
| L-Glutathione | | | | 25 | mg | 5 | mg |
| Lipase | (from pure plant enzymes) | 28 | USP units | 5 | mg | 2.5 | mg |
| L-Isoleucine | | | | 2.5 | mg | | |
| L-Leucine | | | | 2.5 | mg | | |
| L-Lysine | | | | 3.5 | mg | | |
| L-Valine | | | | 2.5 | mg | | |
| Maca Root Extract | | | | 5 | mg | | |
| Magnesium Chloride | | | | | | 2.5 | mg |
| Magnesium Citrate | | 6.67 | mg | 20 | mg | | |
| Malt Diastase | | 1.0 | mg | | | | |
| Maltase | (Malt diastase from pure plant | | | 5 | mg | 2.5 | mg |
| | enzymes) | | | | | | |
| Manganese | | 2.5 | mcg | 10 | mcg | | |
| Marine Phytoplankton | | | | 5 | mg | | |
| Molybdenum | | 2.5 | mcg | | | | |
| Methionine | | | | 2.5 | mg | | |
| Milk Thistle | | 68 | mg | 15 | mg | 1 | mg |
| Morus albus | | | | 25 | mg | | |
| MSM | | 10 | mg | 10 | mg | | |
| Mucuna pruriens | | | | 25 | mg | 1.5 | mg |
| Muira puama | | | | 20 | mg | 1.5 | mg |
| Nettle root extract | Urticaceae urtica | | | 5 | mg | | |
| Olive leaf extract | | 88.89 | mg | 65 | mg | | |
| Omega-3 (Alpha-linolenic acid | (from flaxseed) | 7.5 | mg | 7.5 | mg | | |
| Omega-3 (Docosahexaenoic | (from flaxseed) | 2.5 | | 7.5 | mg | | |
| acid) | | | | | | | |
| Omega-3 (Eicosapentoenoic | | 3.7 | | 7.5 | mg | | |
| acid) | | | | | | | |
| Omega 6 (Gamma-linolenic | (from flaxseed) | 2.5 | mg | | | | |
| Acid) | | | | | | | |
| Omega 6 (Linoleic Acid) | | 3.5 | mg | | | | |
| Omega 9 (Oleic Acid) | | 2.5 | mg | | | | |
| Ornithine | | | | 2.5 | mg | | |
| Ox Bile | | 1.0 | mg | 2.5 | mg | | |
| Pancreatin | | 3.5 | mg | | | | |
| Pantothenic Acid | | 2.2 | mg | | | | |
| Papain | (from pure plant enzymes) | 1.0 | mg | 7.5 | mg | | |
| Pepsin | | 1.0 | mg | | | | |
| Pfaffia paniculta (Suma) | | | | 20 | mg | | |
| Phosphatidylserine | (from soy lecithin)! | | | 3.5 | mg | | |
| Phosphorus | | 2.5 | mcg | 2.5 | mcg | | |
| Polygonum Multiflorum | | | | 10 | mg | 10 | mg |
| Potassium | | 2.5 | mcg | | | 10 | mcg |
| Protease | (from pure plant enzymes) | 36 | USP units | 5.0 | mg | 2.5 | mg |
| Pumpkin Seed Extract | | 5.0 | mg | 5.0 | mg | | |
| Rhodiola rosea | | | | 3.5 | mg | 2.0 | mg |
| Rhododendron Caucasicum | Siberian Rhododendron | | | 5 | mg | | |
| Selenium | | 2.25 | mcg | 5.0 | mcg | 2.0 | mcg |
| Threonine (herbal) | | | | 2.5 | mg | | |
| Tribulus terrestris | | | | 35 | mg | 2.0 | mg |
| Vitamin A | | | | 30 | IU | | |
| Vitamin B12 | | 25 | mcg | 100 | mcg | 10 | mcg |
| Vitamin D | | | | 125 | IU | 100 | IU |
| Vitamin E | | | | 35 | IU | | |

The ingredients of Table I, presented in alphabetical order by either their common or scientific names, are now briefly discussed.

### Acacia Leaf Extract

Acacia is a genus of shrubs and trees belonging to the subfamily Mimosoideae of the family Fabaceae. Acacias, also known as thorntrees or wattles, tend to be thorny and pod bearing. The name derives from ακ└ζ (akis) that is Greek for a sharp point.

There are roughly 1300 species of Acacia worldwide, about 960 of them native to Australia, with the remainder spread around the tropical to warm-temperate regions of both hemispheres, including Africa, southern Asia, and the Americas.

Several Acacia species have important uses in traditional medicine, most all of the uses have been shown to have a scientific basis, since chemical compounds found in the various species have medicinal effects. An extract may be prepared by boiling leaves.

### Acidophilus (Lactobacillus, Bulgarius, Bifidus)

Acidophilus Powder contains acidophilus, bifidus, and bulgarius to balance and encourage the growth of friendly intestinal flora in the large and small intestines, aid dairy digestion, which supports immune response, digestion, assimilation, yeast balance (candida), liver health, detoxification, and the side effects from antibiotic treatment.

Some health professionals also claim that acidophilus helps correct constipation, diarrhea, mucus colitis and diverticulitis, reduces blood cholesterol, enhances the absorption of nutrients, sweetens bad breath, treats acne and other skin disorders, conquers harmful bacteria and certain viruses, helps alleviate candidiasis and other vaginal infections, and may even prevent cancer.

### Alfalfa

Alfalfa (Medicago sativa) is a fiber rich flowering plant in the pea family Fabaceae cultivated as an important forage crop. Alfalfa has been used as an herbal medicine for over 1,500 years. Middle Eastern cultures have long used alfalfa as fodder for horses claiming increased speed and strength of the animals and leading to the name “Al fal fa” meaning “father of all foods. Like other members of the legume family, alfalfa is a fairly good source of protein (up to 50%), B-complex vitamins and several minerals (calcium, magnesium, phosphorous, iron, and potassium). Alfalfa is also high in protein, vitamin A, vitamin C, vitamin D, vitamin E, and vitamin K and is found in modern dietary supplements as an ingredient targeted to lowering cholesterol, increasing energy levels and “detoxifying” the blood. Alfalfa is believed to reduce cholesterol and blood sugar levels.

In addition, alfalfa also contains saponins that, like those found in various ginseng roots, may have adaptogenic or stimulatory actions on the cardiovascular and nervous systems. Alfalfa is also promoted as a “detoxifier” for the liver and bloodstream, possibly due to its alkalizing nature. The isoflavone/phytoestrogen content of alfalfa may explain claims of anticancer activity and benefits in relieving menopausal symptoms, relieving pain and stiffness of arthritis/bursitis, alleviating postmenopausal side effects (hot flashes), increasing energy levels, and reducing fatigue.

### Alpha Lipoic Acid

Alpha-lipoic acid (ALA), also known as lipoic acid (or thioctic acid), is a sulfur containing fatty acid found inside every cell of the human body. The main function of alpha-lipoic acid is to generate the energy required to keep living organisms alive and functioning. Lipoic acid plays a key role in a variety of vital energy-producing reactions in the body that turns glucose (blood glucose) into energy. Alpha-lipoic acid is a potent biological antioxidant, substance that neutralizes potentially harmful chemicals called free radicals. Free radicals are highly reactive chemicals that can damage important parts of cells, including deoxyribonucleic acid (DNA—the molecule inside cells that carries genetic information and passes it from one generation to the next). This damage may play a role in the development of cancer. alpha-lipoic acid has been shown to slow the damage in cells caused by free radicals, and in many cases stabilize or even reverse cell damage. What makes alpha lipoic acid unique, is that it functions in water and fat. Unlike more common antioxidants such as vitamins C and E, it appears to be able to recycle antioxidants such as vitamin C and glutathione after they have been expended. Glutathione is an important antioxidant that helps the body eliminate potentially harmful substances. Alpha lipoic acid increases the formation of glutathione.

Alpha-lipoic acid is said to prevent or treat many age-related diseases, from heart disease, and stroke to diabetes, Parkinson's disease and Alzheimer's disease, as well as declines in energy, muscle strength, brain function, and immunity. Alpha-lipoic acid is also being studied for treatment or prevention of HIV disease and multiple sclerosis.

Alpha lipoic acid is made by the body and can be found in very small amounts in foods such as spinach, broccoli, peas, Brewer's yeast, Brussel sprouts, rice bran, and organ meats.

### Amylase

Amylase is a specific class of digestive enzymes that helps to break down carbohydrates. Specific amylase proteins are designated by different Greek letters. All amylases are glycoside hydrolases and act on α-1,4-glycosidic bonds. Amylase is found in saliva and begins digestion of food as soon as chewing begins in the mouth.

An amylase inhibitor retards the digestion of starch thereby potentially improving postprandial carbohydrate tolerance in people with low glucose tolerance, so as excess dietary carbohydrate is metabolized to fat. Inhibition of carbohydrate digestion may help in weight management.

Astragalus membranaceus

Astragalus membranaceus (Astragalus propinquus) also known as huáng qí is a flowering plant in the family Fabaceae. It is one of the 50 fundamental herbs used in traditional Chinese medicine, often in combination with other herbs, to strengthen the body against diseases such as diabetes. It contains antioxidants, which protect cells from damage caused by free radicals.

In western herbal medicine, Astragalus is primarily considered a tonic for enhancing metabolism and digestion and is consumed as a tea or soup made from the (usually dried) roots of the plant, often in combination with other medicinal herbs. It is also traditionally used to strengthen the immune system and in the healing of wounds and injuries. It has been asserted that Astragalus membranaceus improves the functioning of the lungs, adrenal glands and the gastrointestinal tract, increases metabolism, lowers blood pressure, treats diabetes, protects the liver, induces sweating, promotes healing and reduces fatigue. It is also believed that Astragalus membranaceus may increase the production of interferon and serve to activate immune cells such as macrophages.

Astragalus has antibacterial, anti-inflammatory and diuretic properties. It is sometimes used topically for wound treatment. In addition, studies have shown that astragalus may have anti-viral properties and may stimulate the immune system, suggesting that it is indeed effective in preventing colds. Astragalus stimulates the immune system in several ways, such as boosting the immune system function, enhancing metabolism and digestion, and assisting healing of wounds and injuries.

In the United States, researchers have investigated astragalus as a possible treatment for people whose immune systems have been compromised by chemotherapy or radiation. In these studies, astragalus supplements have been shown to speed recovery and extend life expectancy. Research on using astragalus for people with AIDS has produced inconclusive results.

Recent research in China indicates that Astragalus may offer antioxidant benefits to people with severe forms of heart disease, relieving symptoms and improving heart function. It appears to help signal the white blood cells known as neutrophils to migrate to places they are needed to fight infection. It stimulates the “germ eating” white blood cells known as macrophages, activates T cells and natural killer (NIK) cells, and increases the production of immune globulins. It increases the number of stem cells in bone marrow, and encourages their maturity into active white blood cells. Usually taken with a variety of other herbs, it is said that Astragalus as a whole herb aids in recovery from both cancer and the side effects of cancer therapy, eases chronic respiratory infections, and enhances health in HIV patients.

### Biotin

Biotin (also known as vitamin H or B7), has the chemical formula C₁₀H₁₆N₂O₃S (Biotin; Coenzyme R, Biopeiderm). It is a water-soluble B-complex vitamin that is a cofactor in gluconeogenesis and in the metabolism of fatty acids and leucine.

Biotin works with other B vitamins to make healthy cells, and to convert carbohydrates, fats, and proteins into energy. Biotin is also known as the vitamin that produces healthy hair and helps prevent graying. Biotin is a widely used ingredient in hair products due to its promotional properties. Biotin increases hair cortex elasticity, prevents breakage, and thickens the hair cuticles. A condition called seborrhea dermatitis, or cradle cap, which is characterized by a dry, scaly scalp, may occur as a result of biotin deficiency. Biotin plays a vital role in the production of energy from the metabolism of carbohydrates and fats. It may support the health of nails and offer treatment for brittle nails. Biotin also promotes healthy hair, skin, sweat glands, nerve tissue, bone marrow, and male sex glands.

### Bismuth

Bismuth is a right-sided cell receptor and may be considered essential to human health. Bismuth is biologically associated with gastrointestinal and mental health. While lithium is better known for its therapeutic properties with manic-depressive/bipolar disorders, both exert similar effects on their respective chemical environment: Lithium with regard to a potassium/sodium balance (all right-sided cell receptors), and bismuth with regard to zinc/phosphorus balance (all left-sided cell receptors).

Bismuth deficiency is known to result in low stomach acid levels, particularly lower GI regions. Low Bismuth levels are invariably found in persons with an active infection of the Helicobacter Pylori bacterium, which is responsible for some gastric ulcers and a number of other conditions corresponding to the lower parts of the stomach.

Bismuth, through its antimicrobial action, is more appropriate for peptic involvement to inhibit H.Pylori activity, where it supports an increase in upper stomach acid levels.

### Black Cherry

Wild Black Cherries (Prunus serotina) are native to North America and were extensively used by Native Americans for their therapeutic properties. The fruit was eaten fresh or used in cooking applications, and the bark was believed to have astringent properties. Wild Cherries, like many other fruits, are a naturally rich source of flavonoids. They also contain calcium salts, potassium, lignins and beneficial tannins. Black Cherry extract or Black Cherry concentrate has been found to prevent attacks of gout. Black, sweet yellow, and red sour cherries have all shown to be effective. There have been many additional anecdotal reports of cherry extract as an effective treatment for the pain and inflammation of gout.

### Bromelain

Bromelain typically refers to one of a group of proteolytic enzymes (enzymes capable of breaking down protein) extracted from the plant family Bromeliaceae (e.g., pineapple), or it can refer to a combination of those enzymes along with other compounds produced in an extract. Bromelain is used in treating a large number of medical conditions. It appears to work by blocking some pro-inflammatory metabolites that accelerate and worsen the inflammatory process. Bromelain is a natural agent that supports the body to provide anti-inflammatory benefits and for this reason is helpful in healing minor injuries, particularly sprains and strains, muscle injuries, and the pain, swelling, and tenderness that accompany sports injuries. Also as a result of its natural anti-inflammatory effect, bromelain has been found to dramatically reduce postoperative swelling in controlled human research. Double-blind research has found bromelain effective in reducing swelling, bruising, and pain. The anti-inflammatory effect of bromelain is the probable reason this natural enzyme has been found effective for people suffering from joint pain. Again, probably due to its natural anti-inflammatory action, bromelain was reported to help patients with rheumatoid arthritis in preliminary research. In that trial, in which bromelain was given for varying (3-week to 13-month) periods, 73% had good to excellent results in reducing joint pain.

Cystoseira canariensis

Cystoseira canariensis is a sulfonated polysaccharide from brown seaweed-that binds to myostatin protein. Natural sulfated polysaccharides (SPs) derived from brown seaweed comprise a complex group of macromolecules with a wide range of important physiological properties. SPs have been shown to bind and directly regulate the bioactivity of growth factors and cytokines such as basic fibroblast growth factor, interferon, various enzymes and transforming growth factor. Myostatin is a member of the transforming growth factor-beta (TGF-beta) family that acts as a negative regulator of skeletal muscle mass. SPs isolated from the brown seaweed Cystoseira canariensis have been shown to bind to the myostatin protein in serum.

### Calcium

About 99 percent of the calcium in the human body is held in the bones and teeth. The remaining 1 percent of calcium circulates in the bloodstream, where it performs a variety of important functions. It helps to contract muscles and helps regulate the contractions of the heart. It plays a role in the transmission of nerve impulses and in blood clotting. Calcium is involved in the stimulation of contractions of the uterus during childbirth and in milk production. It also regulates the secretion of various hormones and aids in the functioning of various enzymes within the body. When Calcium is absorbed in the small intestine, it passes from there either into the bloodstream or ultimately into the bones. The most efficient absorption of calcium is dependent on the presence of vitamin D in the body, which is a key ingredient in the various hormones that enable calcium to pass from the digestive system into the blood or bones. Similarly, there are optimal ratios of phosphorus to the amount of calcium consumed that permit calcium to be more completely utilized. Hormonal secretions of the parathyroid and thyroid glands (parathyroid hormone and calcitonin, respectively) also help maintain calcium equilibrium in the blood.

Calcium is responsible for construction, formation and maintenance of bone and teeth. This function helps reduce the occurrence of osteoporosis. Calcium is also a vital component in blood clotting systems and also helps in wound healing. Calcium helps to control blood pressure, nerve transmission, and the release of neurotransmitters. Calcium is an essential component in the production of enzymes and hormones that regulate digestion, energy, and fat metabolism. Calcium helps to transport ions (electrically charged particles) across the membrane. Calcium is essential for muscle contraction. Calcium assists in maintaining all cells and connective tissues in the body. Calcium may be helpful in reducing the incidence of premature heart disease, especially if adequate intakes of magnesium are also maintained. Calcium may help to prevent periodontal disease (gum disease).

### Carnosine

Carnosine (beta-alanyl-L-histidine) is a dipeptide of the amino acids beta-alanine and histidine. It is highly concentrated in muscle and brain tissues of the human body. It is reported that carnosine improved on a measure of socialization and receptive vocabulary in children with autism. The improvements found in this study could have been due to maturation, educational interventions, placebo effect, or other confounds that were not addressed in the study design. Supplemental carnosine may increase corticosterone levels, which can explain the hyperactivity sometimes seen in high doses. Researchers in Britain, South Korea, Russia and other countries have also shown that carnosine has a number of antioxidant properties that may be beneficial.

Carnosine has been proven to scavenge reactive oxygen species (ROS) as well as alpha-beta unsaturated aldehydes formed from peroxidation of cell membrane fatty acids during oxidative stress. It can oppose glycation and it can chelate divalent metal ions. Chronic glycolysis is suspected to accelerate aging.

While a small number of studies have produced evidence of beneficial effects of N-acetyl carnosine in treating cataracts of the eyes, these and other ophthalmologic benefits have not been proven.

Typical vegetarian diets are thought to be lacking in carnosine, but whether this has a detrimental effect on vegetarians is controversial.

Carnosine has been found to inhibit diabetic nephropathy by protecting the podocytes and mesangial cells.

### Cellulase

Cellulase refers to a class of enzymes produced are chiefly by fungi, bacteria, and protozoans that catalyze the hydrolysis (or cellulolysis) of cellulose (i.e., breaks down dietary fiber). Because the body does not produce cellulase, this food enzyme is essential and must be eaten on a daily basis. Only raw foods contain cellulase. Of all the digestive enzymes, a cellulase deficiency produces the most categories of problems. The symptoms of cellulase deficiency can best be described as malabsorption syndrome (impaired absorption of nutrients, vitamins, or minerals from the diet by the lining of the small intestine). Malabsorption has many symptoms including lower abdominal gas, pain, bloating and problems associated with the jejunum and pancreas.

### Chlorella

Chlorella is a genus of single-celled green algae, belonging to the phylum Chlorophyta. Chlorella has a fibrous, indigestible outer shell (20%) and its inner nutrients (80%). This fibrous material is spherical in shape, about 2 to 10 μm in diameter, and is without flagella. Chlorella contains the green photosynthetic pigments chlorophyll-a and -b in its chloroplast. Through photosynthesis it multiplies rapidly requiring only carbon dioxide, water, sunlight, and a small amount of minerals to reproduce.

Numerous research projects in the United States and Europe indicate that chlorella can aid the body in detoxification while strengthening the immune system response. In Japan, interest in chlorella has focused largely on its detoxifying properties. It is also this fibrous material that greatly augments healthy digestion and overall digestive track health. Chlorella plays a particularly crucial role in detoxification through stool. Once detoxification occurs in the intestines, chlorella effectively helps to remove the impurities.

It is the fibrous material that has been proven to actually bind with impurities that can accumulate in the body. A clean bloodstream, with an abundance of red blood cells to carry oxygen, is necessary to a strong natural defense system. Chlorella's cleansing action on the bowel and other elimination channels, as well as its support for the liver, helps keep the blood clean. Clean blood assures that metabolic wastes are efficiently carried away from the tissues.

### Chondroitin Sulfate

Chondroitin sulfate is a sulfated glycosaminoglycan (GAG) composed of a chain of alternating sugars (N-acetylgalactosamine and glucuronic acid). It is usually found attached to proteins as part of a proteoglycan. A chondroitin chain can have over 100 individual sugars, each of which can be sulfated in variable positions and quantities. Understanding the functions of such diversity in chondroitin sulfate and related glycosaminoglycans is a major goal of glycobiology. Chondroitin sulfate is an important structural component of cartilage and provides much of its resistance to compression. Along with glucosamine, chondroitin sulfate has become a widely used dietary supplement for treatment of osteoarthritis.

### Chromium Picolinate

Chromium Picolinate is a combination of the element chromium and picolinic acid. Chromium is a naturally occurring mineral, trace amounts of which are found in everyday foods like meat, poultry, fish, and whole-grain breads. Chromium is important in processing carbohydrates and fats, and helping cells respond properly to insulin, thereby making blood sugar available to the cells Chromium Picolinate is a nutritional supplement that works to increase the efficiency of insulin to optimal levels. Chromium Picolinate has been touted a miracle mineral, one advertised to have myriad effects including weight loss, mood enhancement, energy promotion, increase in life span, and even the prevention of acne. In theory chromium might help prevent heart disease. Chromium is essential for a healthy metabolism, especially as a person ages. A person receiving insufficient chromium may feel run down or have trouble keeping weight off. Taking chromium daily can result in significant health benefits, like increased energy, improved weight control, and better carbohydrate metabolism.

### Chrysin

Chrysin is a naturally occurring flavone chemically extracted from the blue passion flower (Passiflora caerulea). Honeycomb also contains small amounts. Chrysin is advertised as an aromatase inhibitor supplement by bodybuilders and athletes. However, studies done in vivo do not show proof of aromatase inhibitor activity. Since chrysin is available as an herbal supplement, some users, for instance body builders, take chrysin with the hope of raising testosterone levels or stimulating testosterone production.

### Copper

Copper is one of the relatively small group of metallic elements which are essential for human health. These elements, along with amino and fatty acids as well as vitamins, are required for normal metabolic processes to occur. However, as the body cannot synthesize copper; the human diet must supply regular amounts for absorption. Copper is essential for life, which means that the body must have copper to stay healthy. In fact, for a variety of biochemical processes in the body to operate normally, copper must be a part of the diet. Copper is needed for certain critical enzymes to function in the body. These enzymes are involved with energy metabolism—the way that the body generates the energy to function. In addition, copper is involved in the functioning of the nervous system, in maintaining the balance of other useful metals in the body such as zinc and molybdenum, and possibly other functions. Scientists around the globe are continually learning more about the body's need for copper and its benefits in the normal diet. Copper combines with certain proteins to produce enzymes that act as catalysts to help a number of body functions. While some of these enzymes help provide energy required by biochemical reactions, others are involved in the transformation of melanin for pigmentation of the skin. Still others help to form cross-links in collagen and elastin and thereby maintain and repair connective tissues. This function is especially important for the heart and arteries. Too little copper in the body can actually lead to disease. Research suggests that copper deficiency is one factor leading to an increased risk of developing coronary heart disease.

### Co Enzymes QI0 (CoQ10)

CoQ10 is a naturally occurring compound found in every cell in the body. CoQ10's alternate name, ubiquinone, comes from the word ubiquitous, which means “found everywhere.” CoQ10 plays a key role in producing energy in the mitochondria, the part of a cell responsible for the production of energy, in the form of ATP. However, as they have no mitochondria, red blood cells and eye lens cells have no CoQ10.

In each human cell, food energy is converted into energy in the mitochondria with the aid of CoQ10. Ninety-five percent of all the human body's energy requirements (ATP) are converted with the aid of CoQ10. Therefore, those organs with the highest energy requirements—such as the heart, the lungs, and the liver have the highest CoQ10 concentrations. CoQ10 is used by cells to produce energy needed for cell growth and maintenance. CoQ10 is also used by the body as an antioxidant.

General benefits of CoQ10 include: improved efficiency of cellular energy production, enhanced immune system strength, reduction of high blood pressure, improved and sometimes reversed periodontal disease, improved efficacy of weight loss programs, and reduced side effects from chemotherapy.

Cardiovascular Benefits of CoQ10 include increased intrinsic strength of the heart muscle. COQI0 is used in Japan and elsewhere to treat congestive heart failure, cardiac arrhythmia, ischemic injury and angina pectoris. CoQ10 is effective in lowering blood pressure and reducing ischemic & hypoxic injury.

Anti-aging Effects of CoQ10 include increasing energy in sedentary people. CoQ10 also acts as an immunologic stimulant and a potent antioxidant as well as aiding in detoxification. CoQ10 has been shown to dramatically extend the life of female mice.

### Cyanotis Vaga

The herb Cyanotis Vaga is the source of a high potency natural phytochemical, Beta Ecdysterone which is typically standardized to 97%. Until recently Beta Ecdysterone was not readily available because there was no efficient way to extract it. Recently a kilo of pure Beta Ecdysterone sold for $20,000.00. The breakthrough in the technology for invitro cell cultivation provided the purity and concentration to make Beta Ecdysterone economically viable for supplementation. Beta Ecdysterone improve performance, decreases fat and increases lean muscle mass. Scientific research clearly demonstrates the effectiveness of Beta Ecdysterone with no reported adverse side effects. In one study, 117 speed skaters were tested for work capacity, body weight and lung capacity. Beta Ecdysterone supplementation resulted in an increase in all of the aforementioned parameters. In another study conducted by S. Yu. Simakin, 78 highly trained athletes were either given a placebo, protein, or Beta Ecdysterone with a protein supplement. Results were best for the group consuming Ecdysterone with a protein supplement. In this group, a 6 to 7% increase in lean muscle mass was observed after just 10 days, and fat content was reduced by an average of 10% for both males and females. This safe and effective compound is a revolutionary advance in the health, fitness and bodybuilding world. For best results, Beta Ecdysterone is ingested with protein rich meals.

### Dehydroepiandrosterone (DHEA)

Dehydroepiandrosterone (DHEA) is a natural steroid prohormone produced from cholesterol by the adrenal glands, the gonads, adipose tissue, brain and in the skin (by an autocrine mechanism). DHEA is the precursor of androstenedione, which can undergo further conversion to produce the androgen testosterone and the estrogens estrone and estradiol. DHEA is also a potent sigma-1 agonist. The most remarkable finding about Dehydroepiandrosterone comes from a human study by S. S. C. Yen and associates at the University of California, San Diego, in which 50 mg a day of DHEA over a 6-month period restored serum levels of DHEA in both men and women to youthful ranges. DHEA replacement was associated with an increase in perceived physical and psychological well being for both men (67%) and women (84%). Increases in lean body mass and muscle strength were reported in men taking 100 mg a day, but this dose appeared to be excessive for women. DHEA (50 or 100 mg per day) was also shown to significantly elevate insulin growth factor (IGF). Aging typically causes a decline in IGF levels that contribute to the loss of lean body mass, as well as to excess fat accumulation, neurological impairment and age-associated immune dysfunction. Various clinical studies provide evidence that DHEA benefits memory, mood, and electroencephalogram (EEG) readings, and may play a protective role against neurodegenerative diseases. It was shown to prevent pharmacologically induced amnesia and mental impairment by benzodiazepine (Valium-like) drugs.

Epidemiological studies show that low levels of DHEA are associated with the risk of Alzheimer's disease, and a new study provides some molecular mechanisms for how DHEA supplementation may help in part to prevent Alzheimer's disease.

In one study, DHEA inhibited chemically induced cancers in the colon, lung, breast, and skin. When it was applied directly to the skin, it prevented chemically induced cancer. DHEA had this affect by inhibiting the binding of carcinogens to skin cells and by inhibiting the enzyme G6PDH.

Dehydroepiandrosterone demonstrates a striking ability to maintain immune system synchronization. Oral supplementation with low doses of DHEA in aged animals restored immunocompetence to a reasonable level within days of administration. DHEA boosted beneficial interleukin-2 and suppressed levels of damaging interleukin-6 which is overproduced in the aged, contributing to autoimmune disease, immune dysfunction, osteoporosis and reduced healing. Suppression of interleukin-6 with 200 mg a day of DHEA was shown to be effective against systemic lupus erythematosus.

A study of elderly volunteers showed that 100 mg a day of DHEA markedly enhanced the antibody response to the influenza vaccine. In influenza epidemics, 80-90% of mortality occurs in people over age 64. While influenza vaccines can be highly effective in young adults, 30-50% of the elderly fail to generate protective immunity. Elderly people who take an annual flu shot may want to consider taking 50 mg of DHEA daily at least two days before vaccination to help the vaccine induce an immune response. Other benefits of DHEA include ability to protect against heart disease and atherosclerosis. A new study using coronary artery angiography showed that low levels of DHEA are a significant risk factor for coronary artery disease. Another new study showed that DHEA inhibits abnormal blood platelet aggregation, a factor in the development of atherosclerosis, sudden heart attack and stroke. In February 2004, a team of scientists from the University of Wisconsin (Madison) exposed human neural stem cells to DHEA and yielded some of the world's first direct evidence of the biological effects of DHEA on the human nervous system. DHEA significantly increased cellular division in the cells contained in the lab dish, increased the number of neurons produced by the stem cells by up to 29%. The researchers observed that, of all the steroids to which the lab stem cells were exposed, DHEA was the only one to have such a direct effect on stem cell growth and new neuron formation. This new research suggests that DHEA may moderate the production of new brain cells as aging occurs, and when taken collectively with previous broad research suggesting the role of DHEA in enhancing the brain and memory function, an important potential role of DHEA in cognitive dysfunctions such as senile dementia, including Alzheimer's Disease, is revealed.

Daily DHEA supplementation has also recently been shown to remedy some of the side effects of menopause. During a yearlong study in Italy, 20 menopausal women took 25 milligrams of DHEA. Estrogen, testosterone, and dihydrotestosterone increased by between three and four times, while progesterone rose nearly two-fold. Lead researchers Dr. Alessandro Genazzani from the University of Modena (Italy) suggest that one day, DHEA may replace hormone replacement therapy (HRT), as this experiment resulted with all women reporting improvements in menopausal symptoms such as hot flashes, without any side effects of any kind.

DHEA recently has also been found to reduce risk factors associated with age-related heart disease. DHEA improves blood vessels by stimulating the linings of arteries to produce nitric oxide, a substance that relaxes arterial tension. In a Japanese study of 24 men, average age 54, with elevated cholesterol, Kumamoto University School of Medicine researchers found that 25 mg of DHEA daily benefited endothelial function (flexibility of arteries), insulin sensitivity, and fibrinolytic activity, with changes taking place as early as within four weeks of supplementation. DHEA for men and women, in youth or in older age, can be considered as a safe, multi-modal anti-aging supplement promoting quality of life and increasing the odds of living longer lives.

Emblica officinalis (Indian gooseberry)

The Indian gooseberry (Phyllanthus emblica, syn. Emblica officinalis) is a deciduous tree of the Euphorbiaceae family. It is known for its edible fruit of the same name. Indian gooseberry has undergone preliminary research, demonstrating in vitro antiviral and antimicrobial properties. Experimental preparations of leaves, bark or fruit have shown potential efficacy against laboratory models of disease, such as for inflammation, cancer, age-related renal disease, and diabetes.

A human pilot study demonstrated reduction of blood cholesterol levels in both normal and hypercholesterolemic men.

### Ester-C

For some people, vitamin C is associated with gastrointestinal discomfort, especially in large doses. This is due in large part to the acidity of ascorbic acid itself. Ester C's manufacturing process neutralizes the acidity of ascorbic acid to provide the nutritional benefits of both vitamin C and essential minerals in a form that is gentle to the stomach.

Vitamin C, America's most popular vitamin also is one of the most versatile nutrients. It is best known for its role in providing cold weather season protection. Vitamin C, a potent antioxidant, helps prevent free radical damage and may support the body's natural defenses. Millions of people worldwide became aware of the benefits of vitamin C due to the influence of the late Nobel Prize winning scientist, Dr. Linus Pauling, who advocated generous dosages of vitamin C supplements.

Vitamin C, or ascorbic acid, was first identified as essential because of its role in the prevention of scurvy, a classic nutrient deficiency disease. Scurvy is the total breakdown of collagen, the connective protein that binds tissue together. Although scurvy is rare in modern industrial society, vitamin C still is associated with support for joint function.

Vitamin C is also associated with promoting capillary health and aiding in the normal development and maintenance of bones, cartilage and teeth.

### Fucoxanthin

Fucoxanthin is a carotenoid, with formula C₄₂H₅₈O₆. It is found as an accessory pigment in the chloroplasts of brown algae and most other heterokonts, giving them a brown or olive-green color. Some metabolic and nutritional studies carried out at Hokkaido University indicate that fucoxanthin promotes fat burning within fat cells in white adipose tissue by increasing the expression of thermogenin.

Fucoxanthin reduces fat because of its increase in cell energy expenditure. This is important because fucoxanthin helps to increase the metabolic rate, which becomes progressively slower as you age, explaining why normal dieting doesn't always have the desired long-term results. There have been recent studies in both humans and animals that showed taking fucoxanthin supplements helps to reduce body weight as well as significant losses in visceral fat, decrease in liver and abdominal fat, and an increase in energy expenditure. Subjects taking the fucoxanthin supplements lost an average of 14 pounds, 11 pounds more than the placebo group.

Ganoderma lucidum

Ganoderma lucidum is a fungus known by its many names like “Reishi,” “Ling zhi,” and Mannentake,” among others, which has been recognized for hundreds or even thousands of years as a powerful medicinal fungi because it has properties often associated with health and healing, long life, knowledge, and happiness. In fact, during the ancient time, it is believed that the Ganoderma lucidum in medicine was considered so promising that its medicinal value has been attested to in a 2,000 year old Chinese medical text known as an authentic textbook of Oriental medical science. For years, medicinal mushrooms such as Ganoderma lucidum have long been included in the history of folk medicine for contributing much to curing cancers of all sorts and for showing positive effects to the development of the immune system.

Believed by the Chinese as the “Miraculous King of Herbs”, Ganoderma lucidum is highly regarded for its medicinal properties that help improve the human body's healing ability while helping its user to maintain good physical shape. Aside from promoting longevity, Ganoderma lucidum has unique properties that contribute much to the strengthening of the immune system. Somewhat rare in the West, Ganoderma lucidum has been worshipped as a kind of herbal medicine the emperors of the great Japanese and Chinese dynasties drank with their special teas and mushroom concoctions to achieve greater vitality and longer life. Ganoderma lucidum was also believed to be visible in the “elixir of eternal youth” for which the ancient Taoists constantly searched. Aside from contributing much to the treatment of various diseases, Ganoderma lucidum has also become popular because of its promising properties that might extend the life span while increasing vigor and vitality.

Ginkgo biloba

Ginkgo biloba is a unique species of tree with no close living relatives. The extract of the Ginkgo leaves contains flavonoid glycosides and terpenoids (ginkgolides, bilobalides) and has been used pharmaceutically. It has many alleged nootropic properties, and is mainly used as memory and concentration enhancer, and anti-vertigo agent.

Ginkgo biloba o extract is believed to have three effects on the human body: improvement in blood flow (including microcirculation in small capillaries) to most tissues and organs; protection against oxidative cell damage from free radicals; and blockage of many of the effects of platelet-activating factor (platelet aggregation, blood clotting) that have been related to the development of a number of cardiovascular, renal, respiratory and central nervous system disorders. Ginkgo can be used for intermittent claudication.

Ginkgo biloba is also believed to improve attention in healthy individuals. It is also being studied as a possible treatment of Alzheimer's disease.

Ginkgo biloba has been used medicinally for thousands of years and is one of the top selling herbs in the United States.

Ginkgo biloba is used for the treatment of numerous conditions, many of which are under scientific investigation. Available evidence demonstrates ginkgo's efficacy in the management of intermittent claudication, Alzheimer's/multi-infarct dementia, and “cerebral insufficiency” (a syndrome thought to be secondary to atherosclerotic disease, characterized by impaired concentration, confusion, decreased physical performance, fatigue, headache, dizziness, depression, and anxiety).

There is promising early evidence favoring the use of Ginkgo biloba for memory enhancement in healthy subjects, altitude (mountain) sickness, symptoms of premenstrual syndrome (PMS), and reduction of chemotherapy-induced end-organ vascular damage.

### Ginseng (Panax Ginseng)

As an adaptogen, Ginseng benefits energy, improves overall health, stimulates immune function, and normalizes glucose levels for diabetics. As a tonic, Ginseng is good for antiaging and longevity. Panax Ginseng is an antiaging adaptogenic herb. Adaptogens maintain homeostasis, increase energy and endurance and enhance sexual performance. They enhance the overall immunity including antibody response, natural killer cell activity and interferon production. The active ingredients in Ginseng are called ginsenosides. Ginsenosides have a variety of effects, ranging from stimulation of the nervous system to reduction of blood sugar levels. Panax Ginseng also functions as antioxidant, stimulates the immune system, thins the blood and reduces “bad” cholesterol levels. Uses and benefits of Ginseng Root Adaptogenic herbs support all the major systems: nervous system, hormonal system and immune system. By normalizing bodily functions, adaptogens help maintain the stable internal environment known as homeostasis. In order for the body parts to work efficiently, the concentrations of water, food substances and oxygen (as well as the conditions of heat and pressure) must remain within certain very narrow limits. The genus name of ginseng “Panax” is derived from the Greek pan (all) akos (cure), meaning “cure-all”. This alone tells a lot about this herb: no single herb can be considered a panacea but ginseng comes close to it. Ginseng is a tonic herb, or an adaptogen that helps to improve overall health and restore the body to balance, and helps the body to heal by itself. Ginseng has been used for centuries to boost energy, sharpen the mind, reduce stress, treat impotence, and extend life. Other traditional uses include: to enhance the immune system, control blood pressure, regulate blood sugar levels, and strengthen the cardiovascular system. By helping body parts remain in balance, adaptogens can help us have more energy and greater health. It will, when the body needs it, be calming and help relieve stress, mental fatigue and weakness. It is beneficial to blood pressure (high or low) as an antidote to many toxic drugs and chemicals, it protects the body against radiation, helps vision, hearing, reduces irritability, and produces strength to the adrenal glands. Known in the Orient as king of the herbs, Korean Ginseng is becoming popular in this country also. It is known for giving energy and endurance.

Some areas of potential benefits: Adrenal glands, age spots, blood pressure, depression, endurance, hemorrhage, longevity, sexual stimulation, stress.

### Glucosamine

Glucosamine (C₆H₁₃NO₅) is an amino sugar and a prominent precursor in the biochemical synthesis of glycosylated proteins and lipids. A type of glucosamine forms chitin, which composes the exoskeletons of crustaceans and other arthropods, cell walls in fungi and many higher organisms. Glucosamine is one of the most abundant monosaccharides. It is produced commercially by the hydrolysis of crustacean exoskeletons or, less commonly and more expensive to the consumer, by fermentation of a grain such as corn or wheat. Glucosamine is commonly used as a treatment for osteoarthritis, as an oral supplement as necessary.

Since glucosamine is a precursor for glycosaminoglycans, and glycosaminoglycans are a major component of joint cartilage, supplemental glucosamine may help to rebuild cartilage and treat arthritis.

### Glycine

Glycine (often abbreviated as Gly or G) is an organic compound with the formula NH₂CH₂COOH. It is the smallest of the 20 amino acids commonly found in proteins. Because it has specialized structural properties in the protein architecture, this compact amino acid is often evolutionarily conserved. For example, cytochrome c, myoglobin, and hemoglobin all contain conserved glycines. Glycine is unique among the proteinogenic amino acids in that it is not chiral. Most proteins contain only small quantities of glycine. A notable exception is collagen, which contains about 35% glycine.

Glycine is used to help create muscle tissue and convert glucose into energy. It is also essential for maintaining healthy central nervous and digestive systems, and has recently been shown to provide protection via antioxidants from some types of cancer.

Glycine is used in the body to help construct normal DNA and RNA strands—the genetic material needed for proper cellular function and formation. It helps prevent the breakdown of muscle by boosting the body's levels of creatine, a compound that helps build muscle mass. High concentrations of glycine are found not only in the muscles, but in the skin and other connective tissues as well. Without glycine the body would not be able to repair damaged tissues; the skin would become slack as it succumbed to UV rays, oxidation, and free radical damage, and wounds would never heal.

Glycine is considered a glucogenic amino acid, which means it helps supply the body with glucose needed for energy. It helps regulate blood sugar levels, and thus glycine supplementation may be useful for treating symptoms characterized by low energy and fatigue, such as hypoglycemia, anemia, and Chronic Fatigue Syndrome (CFS). Glycine is essential for a healthy, normally functioning digestive system. It helps regulate the synthesis of the bile acid used to digest fats, and is included in many commercial gastric antacid agents.

### Gold

Gold is one of the best and most efficient conductors of electrons. The brain and nervous system depend on the movement of electrons between cells to function and in the experience of many who have supplemented with gold, the electrolytic properties of gold are unparalleled in the in the enhancement of that function. Many people are now using this dietary supplement to uplift the emotions and reduce the effects of depression, as well as to enhance the ability to focus. Gold is non-toxic, promotes a general euphoric feeling of well being, stimulates the body's restorative functions, enhances the body's natural defenses against illness, and promotes vitality and longevity.

Grape seed extract

Grape Seed Extract (GSE) provides protection against pollution and environmental toxins. As nature's most potent antioxidant, GSE helps to reduce and prevent free radical damage. It also reduces the rate of aging and wrinkling of the skin, and reduces the risk of cancer.

GSE helps with age related illnesses. GSE improves joint flexibility and prostate function, reduces mental aging and enhances energy.

GSE benefits autoimmune diseases by enhancing the immune system, reducing inflammation, helping to control allergic responses and balancing the breakdown of proteins.

GSE benefits nutritional deficiencies reducing fatigue, improving visual acuity, reducing the frequency and severity of colds and enhancing the benefits of vitamins.

The most significant benefit of GSE is its protection against cardiovascular disease. GSE not only reduces the risk of heart disease and stroke, but also helps improve peripheral and central circulation, reduces varicose veins, repairs artery and vascular lining and aids in resistance to bruising.

Guarana Seed Extract (Paullinia cupana) Guarana (Paullinia cupana (syn. Paullinia crysan, Paullinia sorbilis), is a climbing plant in the Sapindaceae family, native to the Amazon basin and especially common in Brazil. Guarana features large leaves and clusters of flowers, and is best known for its fruit, which is about the size of a coffee berry. Each fruit harbors one seed which contains approximately five times as much caffeine as coffee beans.

Guarana is used in sweetened or carbonated soft drinks and energy shots, an ingredient of herbal tea or contained in capsules. Brazil, which is the third-largest consumer of soft drinks in the world, produces several soft drink brands from guarana extract.

Because guaranine (from guarana seeds) is chemically equivalent to caffeine, guarana is of interest for its potential effects on cognition. In rats, guarana increased memory retention and physical endurance when compared with a placebo. A 2007 human pilot study assessed acute behavioral effects to four doses (37.5 mg, 75 mg, 150 mg and 300 mg) of guarana extract. Memory, alertness and mood were increased by the two lower doses, confirming previous results of cognitive improvement following 75 mg guarana.

Guarana extract reduced aggregation of rabbit platelets by up to 37 percent below control values and decreased platelet thromboxane formation from arachidonic acid by 78 percent below control values.

### Herbal Threonine

Threonine is an important part of many proteins in the body and is necessary for the formation of tooth enamel, elastin, and collagen, which are needed for both healthy skin and wound healing. Threonine has a mild glucose-sparing effect and is useful in the stabilization of blood sugar because it can be converted into glucose in the liver by the process of gluconeogenesis. Threonine is one of the immune-stimulating nutrients (cysteine, lysine, alanine, and aspartic acid are others), as it promotes thymus growth and activity. Threonine may enhance immunity by assisting in the production of agents that fight viral infections. It also can probably promote cell immune defense function.

Threonine is important in the formation of collagen and elastin. When combined with aspartic acid, threonine aids liver and lipotropic function. Threonine is used to treat indigestion and intestinal malfunctions. Threonine may prevent fat from accumulating in the liver. A fatty liver can affect liver function and is associated with diseases of the liver such as cirrhosis. Threonine is an immunostimulant, which promotes the growth of thymus gland.

### Hemicellulase

Hemicellulase is a mixture of enzymes that hydrolyze the indigestible components of plant fibers. Since humans lack the endogenous enzymes required to digest plant fibers, the supplementation of hemicellulase provides humans with an additional source of nutrition and reduces the bulking effect of fibrous foods. Scientific evidence suggests that carbohydrolytic enzymes, such as hemicellulase, can be useful supplements for digestive support and general nutritional support.

### Horse Chestnut Seed Extract

Horse chestnut seed extract is most studied for a condition called chronic venous insufficiency. This term is more common in Europe than the United States. It describes several different problems that may be caused by the failure of lower leg veins to work correctly. These problems include leg swelling (edema), pain, itching, varicose veins, breakdown of skin and skin ulcers. Multiple studies suggest that horse chestnut seed extract may help these problems, possibly as well as other treatments, such as compression stockings.

### Iodine

Iodine is a chemical element used in pharmaceuticals, antiseptics, medicine, food supplements, dyes, catalysts, halogen lights, photography, water purifying, and starch detection. Tincture of iodine (10% elemental iodine in ethanol base) is an essential component of any emergency survival kit, used both to disinfect wounds and to sanitize surface water for drinking. Alcohol-free iodine solutions such as Lugol's iodine, as well as other iodophor type antiseptics, are also available as effective elemental iodine sources for this purpose.

The United States Recommended Daily Allowance (RDA) is 150 micrograms per day for both men and women. Natural sources of iodine include sea life, such as kelp and certain seafood, as well as plants grown on iodine-rich soil. Iodized salt is fortified with iodine. As of 2000, the median intake of iodine from food in the United States was 240 to 300 μg/day for men and 190 to 210 μg/day for women.

In areas where there is little iodine in the diet, typically remote inland areas and semi-arid equatorial climates where no marine foods are eaten, iodine deficiency gives rise to hypothyroidism, symptoms of which are extreme fatigue, goitre, mental slowing, depression, weight gain, and low basal body temperatures.

Iodine deficiency is the leading cause of preventable mental retardation, a result which occurs primarily when babies or small children are rendered hypothyroidic by a lack of the element. The addition of iodine to table salt has largely eliminated this problem in the wealthier nations, but as of March 2006, iodine deficiency remained a serious public health problem in the developing world. Iodine deficiency is also a problem in certain areas of Europe. In Germany it has been estimated to cause a billion dollars in healthcare costs per year.

### Lactase

Lactase (LCT), a member of the β-galactosidase family of enzymes, is a glycoside hydrolase involved in the hydrolysis of the disaccharide lactose into constituent galactose and glucose monomers. In humans, lactase is present predominantly along the brush border membrane of the differentiated enterocytes lining the villi of the small intestine.

Lactase is essential for digestive hydrolysis of lactose in milk. Deficiency of the enzyme causes lactose intolerance.

### L-Arginine

L-arginine is an amino acid that has numerous functions in the body. It helps the body get rid of ammonia (a waste product), is used to make compounds in the body such creatine, L-glutamate, and L-proline, and can be converted to glucose and glycogen if needed. In the body, L-arginine is used to make nitric oxide, which reduces blood vessel stiffness, increases blood flow, and improves blood vessel function.

Dietary supplements that contain L-arginine are recommended to promote heart health, cardiac muscle and vascular function and to prevent “platelet aggregation” (the risk of blood clots) and to lower blood pressure. The Department of Cardiac Surgery in Lodz, Poland released the results of their research into L-arginine function in 2006. According to their data, it is believed that L-arginine improves blood flow to and from the heart by stimulating nitric oxide release. Nitric oxide dilates or enlarges the blood vessels.

Preliminary studies have found that L-arginine may help with conditions that improve when blood vessels are relaxed (called vasodilation), such as atherosclerosis, erectile dysfunction, and intermittent claudication. L-arginine has been used for erectile dysfunction. Like the drug sildenafil citrate (Viagra), L-arginine is thought to enhance the action of nitric oxide, which relaxes muscles surrounding blood vessels supplying the penis. As a result, blood vessels in the penis dilate, increasing blood flow, which helps maintain an erection. The difference in how they work is that Viagra blocks an enzyme called PDE5 which destroys nitric oxide and L-arginine is used to make nitric oxide. In one study, 50 men with erectile dysfunction took either 5 grams of L-arginine per day or a placebo. After six weeks, more men in the L-arginine group had an improvement compared to those taking the placebo. L-arginine's possible activity in wound repair may be due to its role in the formation of L-proline, an important amino acid that is essential for the synthesis of collagen. L-arginine is also used for high blood pressure, migraines, sexual dysfunction in women, and interstitial cystitis.

### L-Glutathione

L-Glutathione is a dominant anti-oxidant enzyme that is water-soluble. It is absorbed mostly in the liver where it helps to fight free radical damage. Free radical damage is harmful to ones health. L-Glutathione is involved in a variety of other functions within the body.

L-Glutathione works in DNA Synthesis and repair, protein and prostaglandin synthesis, and amino acid transport. It assists in the metabolism of carcinogens and toxins. Immune system functioning is enhanced with the use of L-Glutathione, and it aids in the prevention of oxidative cell damage, as well as enzyme activation. Glutathione also helps and maintains the functions of other antioxidants.

There is the possibility of a deficiency of L-Glutathione, especially in an aging person. For example, it is seen in age related macular degeneration, diabetes, as well as lung and gastrointestinal diseases. It can be the cause of pre-eclampsia, Parkinson's, AIDS, and other neurodegenerative diseases.

Lily extract (Clintonia borealis)

Lily extract is a plant extract that has been found, in laboratory tests, to inhibit tumor growth. It could be used to target some of the cancers that are currently most difficult to treat, suggests the research.

Studies suggest that cyclopamine, a chemical extracted from the corn lily, shrinks tumors both in tests on mice and on human cells in vitro. Such findings back previous research demonstrating cyclopamine's action against cancer. A study by Dr Philip Beachy and colleagues at the John Hopkins School of Medicine recently found that cyclopamine effectively killed cultured mouse medulloblastoma cells and tumors implanted in animals, as well as medulloblastoma cells extracted from human tumors. Medulloblastoma is an aggressive brain cancer, which affects children, but currently is difficult to treat.

Researchers believe that cyclopamine blocks the Hedgehog signaling pathway, known to be critical for the growth and differentiation of cells during embryonic development but also implicated in malignancy of tumor cells if activated later in life.

### Lipase

Lipase is an enzyme necessary for the absorption and digestion of nutrients in the intestines. This digestive enzyme is responsible for breaking down lipids (fats), in particular triglycerides, which are fatty substances in the body that come from fat in the diet. Once broken down into smaller components, triglycerides are more easily absorbed in the intestines. Lipase is primarily produced in the pancreas but is also produced in the mouth and stomach. Along with lipase, the pancreas secretes insulin and glucagons; hormones that the body needs to break down sugar in the bloodstream.

### L-Isoleucine

L-Isoleucine is one of three branched chain amino acids (BCAAs), the other two being L-Leucine and L-Valine that are found in proteins of all forms of life. They can be obtained in the diet through animal and vegetable sources. L-Isoleucine is important in hemoglobulin synthesis and in the regulation of blood sugar and energy levels. It also seems to increases endurace.

L-Isoleucine is an isomer of L-Leucine and is metabolized in muscle tissue. Rich sources of L-isoleucine include cashews, almonds, and soy protein. L-Isoleucine is popular among athletes. It is typically taken or used with the other two BCAAs, L-Leucine and L-Valine.

### L-Leucine

L-Leucine is an essential branched-chain amino acid (BCAA) that's broken down in fat structures. L-Leucine cannot be made by the body, and must be acquired through food or dietary supplements. It can be found in nuts, brown rice, and whole wheat bread products. L-Leucine comprises about eight percent of the total amino acid count in the human body's protein structure and is the fourth most concentrated amino acid in skeletal muscle tissue. L-Leucine is essential to basic health. It has athletic applications and appears to have many beneficial effects on sports performance. It helps preserve lean muscle tissue, it supplies the body with energy when under stress (i.e. when engaging in athletic activity), it preserves muscle glycogen (glucose stored in muscle tissue used to power muscular contraction), it maintains nitrogen balance and it enhances thinking abilities that can decline as physical activity becomes more intense. The effects of L-Leucine in the diet are profound. As the strongest of the BCAA's, L-leucine is what's known as a “limiting nutrient”—meaning there must be enough L-Leucine in proportion to other amino acids in order for the body to make use of what is eaten. Simply stated: an L-Leucine deficiency renders the body incapable of making use of the protein that is provided, no matter how much protein is consumed. Without enough L-Leucine, the money spent on quality food and dietary supplements maybe wasted. To make the most of what is eaten, two parts L-Leucine and two parts L-Valine are required for every one part of L-Isoleucine. Optimal results do not occur without meeting this exact ratio.

### L-Lysine

Lysine is one of eight essential amino acids (i.e., building blocks of protein) that the body cannot manufacture on its own. L-Lysine is a specific configuration of the lysine molecule often used in dietary supplements. As a building block of protein, L-Lysine benefits the body by contributing to growth. L-Lysine benefits also include production of carnitine, a substance that converts fatty acids into energy and lowers levels of LDL (“bad cholesterol”) in the bloodstream. In addition, L-Lysine benefits the skeletal system by contributing to the production of collagen, the protein used to make bone, tendons, cartilage and connective tissue. Calcium absorption and use is also facilitated by L-Lysine, and L-Lysine benefits the skin by helping to maintain its health and elasticity.

Without enough L-Lysine in the diet, a range of health conditions may result. Though most people can easily obtain sufficient amounts L-Lysine through diet, those who eat large amounts of sugary cereals and baked good may minimize L-Lysine benefits. This is because L-Lysine is involved in the browning of sugar, which occurs when the two substances bind together. As a result, it is difficult for the body to absorb the L-Lysine in sugary in sugary foods. So, in cases where a person's diet is high in foods that contain a lot of simple sugars, L-Lysine supplements may be of use.

There may be other L-Lysine benefits from supplements for people with specific health concerns. For example, when taken with vitamin C, L-Lysine appears to help symptoms of the heart condition angina pectoris. It has also been used to treat sores caused by the herpes simplex and herpes zoster viruses. Additionally, it is believed that L-Lysine benefits people with osteoporosis by helping to prevent bone loss. As mentioned before, calcium absorption appears to be enhanced by L-Lysine.

Also, since low levels of L-Lysine have been associated with depression, chronic viral infections, asthma, and low levels of thyroid hormones, there are possible L-Lysine benefits for people with those conditions as well. Further. L-Lysine appears to work with equal amounts of the amino acid arginine to build muscle.

### L-Valine

L-Valine is one of three Branched Chain Amino Acids (BCAAs). The other two are L-Leucine and L-Isoleucine discussed hereinabove. These amino acids contribute to the functioning of everyday life. The BCAAs are typically formulated together to provide maximum support to the body. L-Valine is derived from animal and vegetable protein.

L-Valine is involved in glucose metabolism, protein synthesis, and the regulation of the immune system. As with the other BCAAs, L-Valine could be involved in muscle growth and tissue repair, and stimulant activity. There is some evidence that L-Valine is involved in muscle metabolism, the maintenance of nitrogen balance in the body, and exercise recovery time.

### Maca Root Extract

The South American country of Peru is home to numerous beneficial plants, including Peruvian Maca root, a legendary sex-enhancing root passed down from the Inca.

Maca, Lepidium meyenii, is an annual plant that produces a radish-like root. Today, Maca's popularity is very much on the increase, as people discover that the plant really does boost libido, sexual function and overall energy.

### Magnesium (Magnesium Chloride)

Magnesium (Mg) is the fourth most abundant mineral in the body and is essential to good health. Approximately 50% of total body magnesium is found in bone. The other half is found predominantly inside cells of body tissues and organs. Only 1% of magnesium is found in blood, but the body works very hard to keep blood levels of magnesium constant.

Magnesium is needed for more than 300 biochemical reactions in the body as it activates 76 percent of the enzymes in the body. It helps maintain normal muscle and nerve function, keeps heart rhythm steady, supports a healthy immune system, and keeps bones strong. Magnesium also helps regulate blood sugar levels, promote normal blood pressure, and is known to be involved in energy metabolism and protein synthesis. There is an increased interest in the role of magnesium in preventing and managing disorders such as hypertension, cardiovascular disease, and diabetes. Dietary magnesium is absorbed in the small intestines. Magnesium is excreted through the kidneys

Hypomagnesaemia is considered to be as prevalent as 80% in persons with long standing fatigue or environmental toxin damage. Without its presence muscles may not properly relax, and spasticity and tissue damage ensue. It works best when sufficient B6 and zinc are used together with it, particularly in reactive states where lowered immunity is suspected. Magnesium Aspartate in generally very well absorbed and tolerated by the highly sensitive individual. The average American consumes only 40 percent of the recommended daily allowance of magnesium. This has serious consequences, including death, in many people. Potassium is primarily concerned with the way the body uses calcium and sodium. Every doctor knows about the dangers of potassium deficiency, but few recognize that almost half of the patients with a potassium deficiency will also be depleted of magnesium. In fact, the low potassium state often cannot be easily corrected unless magnesium is also given.

Magnesium is useful in preventing unwanted calcification in the kidney, bladder, and joints. If a diet is high in phosphorus (common in many meat dishes as lunch meats, hot dogs, etc. and also in soda drinks), the phosphate binds up the magnesium into magnesium phosphate, which isn't absorbed.

In disease and stress states, more magnesium is needed. If a person is using diuretics (water pills), he/she should make sure his magnesium intake is adequate. Potassium supplementation is usually needed also. The higher the protein consumed the more magnesium is needed. When large amounts of calcium are consumed, more magnesium is also required.

In the formulation of the present invention, Magnesium Chloride is chosen as the Magnesium source.

Magnesium Citrate (see also Magnesium Chloride)

Magnesium is one of the body's most important minerals. It is required as a co-factor in hundreds of enzymatic processes within cells. It helps maintain normal muscle and nerve function, keeps heart rhythm steady, supports a healthy immune system, and keeps bones strong. Magnesium also helps to maintain blood sugar and blood pressure levels already within normal range, and is known to be involved in energy metabolism and protein synthesis.

Magnesium is a major factor in relaxing the smooth muscles within the blood vessels, thereby reducing peripheral vascular resistance and promoting a healthy cardiovascular system. Magnesium also affects circulating levels of norepinephrine and the synthesis of serotonin and nitric oxide.

In bone mineral health, magnesium (or lack of) influences the bone mineral matrix and its ability to metabolize minerals needed for repair and rebuilding. The scientific literature documents the need for a wide range of minerals that are vital to maintaining strong, healthy bones.

A review of assimilation studies suggests that the citrate salt of magnesium is the best absorbed into the bloodstream. The major down-side of using magnesium citrate, however, is that it is only 16% magnesium, and the recommended intake of elemental magnesium to maintain vascular health is 300 mg to 800 mg a day.

Maltase (Malt diastase)

Malt diastase is a carbohydrolytic enzyme useful for digestive support and general nutrition support. Malt diastase is characterized by the ability to break down amylose and other polysaccharides. The enzyme works with amylase and glucoamylase to digest carbohydrate rich foods such as grains as well as malt, maltose, and sugars. Malt diastase is also known as maltase. It is produced by the cells lining the small intestine.

The efficacy or potency of malt diastase is measured in degrees of Diastatic Power (DP), The Diastatic Power (DP), also called the “diastatic activity” or “enzymatic power”, of a grain generally refers only to malts and grains which have begun to germinate. Diastatic power for a grain is measured in degrees Lintner (° Lintner or ° L, or in Europe by Windisch-Kolbach units (° WK).

The act of germination includes the production of a number of enzymes such as amylase which convert starch into sugar; thereby, sugars can be extracted from the barley's (i.e., malt from barley) own starches simply by soaking the grain in water at a controlled temperature: this is called mashing. Other enzymes break long proteins into short ones and accomplish other important tasks.

### Manganese

Manganese is an essential trace mineral in human life. One of the most important roles manganese plays is that of an antioxidant. Antioxidants are necessary to combat the potentially damaging effects of free radicals. Manganese, which has antioxidant, free-radical-fighting properties, is important for proper food digestion and for normal bone structure. It is also has important biological functions such as for proper growth in human body, proper formation of bones and cartilage, and the brain function. Because of its role in mucopolysaccharide synthesis, manganese is important in skeletal and connective tissue development. Manganese is stored in the bones, liver, kidney, and pancreas.

### Marine Phytoplankton

Marine phytoplankton have been an important part of our ocean health for millions and millions of years and have changed life as we know it. Marine microalgae create the majority of the oxygen in our atmosphere and when they're spent they form the sediments at the ocean bottom. Though amazingly small individual cells their vast numbers greatly influence the world's climate. Plankton Research continues on a global basis gathering plankton data to monitor marine life and the overall health of the oceans. Sometimes referred to as “Mother Earth's Milk”, phytoplankton is now available for all mankind to benefit from in a natural Marine Phytoplankton formulation. There is good reason why biologists study these simple marine organisms and ultimately are focused upon solving the earth's human medical problems by studying simple sea creatures. The discovery of marine phytoplankton for human consumption may very well turn out to be the most important discovery in the history of nutrition. An amazing free floating single cell plant which is invisible to the human eye but so important that every living being in the ocean depends on its health and abundance for survival. The harvesting of the marine phytoplankton is not an easy task. In order to do it properly you need the right conditions. A fluctuation of 5 degrees colder or warmer could mean damage to the algae cell or death of the cell. Also if the lighting conditions are not perfect the algae can lose a lot or all of its nutritional integrity. It is well known that the food chain begins with plant species in the aqua environment with phytoplankton at the forefront. Phytoplankton, microscopic marine plants are essentially photosynthetic organisms that synthesize organic substances from an external energy source. These microscopic sea plants are super concentrated in a green pigment called chlorophyll. Photosynthesis, the process of converting simple inorganic chemicals into complex organic substances, is a critical part of energy production and recycling. The end result of photosynthesis is the production of energy nutrients such as carbohydrates, proteins, and fats from sunlight, carbon dioxide and water. Energy nutrients are critical to human health. Phytoplankton contains almost every element need for sustaining human life. It contains nine essential amino acids, essential fatty acids including Omega 3 & 6, Vitamins A, B, C, and D, macro and trace minerals are all contained in phytoplankton all in a balanced natural bioavailable form.

### Methionine

Methionine is an essential, sulphur-containing amino acid, it is required in the body for making nucleic acid, collagen, and protein synthesis. The body can make cysteine, another amino acid from methionine but not vice versa. Methionine can also make choline, a vitamin of the B complex group, in the body with the aid of Vitamin B12 and folacin, provided the diet is high in protein. Methionine acts as an antioxidant, and as such, removes harmful free radicals from the body.

Of all the amino acids, methionine is of key importance for the health of the liver. It protects the liver from fatty degeneration. The cysteine that it forms prevents the destruction of the liver cells. This amino acid helps dissolve cholesterol and assimilates fat. It is required by the pancreas, lymph nodes, and the spleen, and is essential for the synthesis of hemoglobin. It is necessary for the maintenance of normal body weight and also helps to maintain the proper nitrogen balance in the body. Studies show that methionine and choline prevent tumor formation.

### Milk Thistle

Milk thistle helps relieve the symptoms of hepatitis, cirrhosis, and inflammatory liver conditions. It is one of the most effective herbs known for relieving liver disorders. Many herbalists and medical practitioners believe that milk thistle can help protect the liver from damage caused by alcohol, chemicals, drugs, diseases, and toxic plants. Milk thistle contains a chemical substance, silymarin, which is purported to protect liver cells in several ways. First, Silymarin has antioxidant properties. Silymarin also has anti-inflammatory effects, which keeps liver cells from swelling in response to injury. Third, silymarin seems to encourage liver cell growth. Fourth, it prevents certain toxins from getting into liver cells by changing the outside layer of liver cells.

### Molybdenum

Molybdenum is an element classed as a transition metal. The most important use of the molybdenum atom in living organisms is as a metal hetero-atom at the active site in certain enzymes. In nitrogen fixation in certain bacteria, the nitrogenase enzyme which is involved in the terminal step of reducing molecular nitrogen, usually contains molybdenum in the active site (though replacement of Mo with iron or vanadium is known).

Though molybdenum forms compounds with various organic molecules, including carbohydrates and amino acids, it is transported throughout the human body as MoO42-. Molybdenum is present in approximately 20 enzymes in animals, including aldehyde oxidase, sulfite oxidase, xanthine oxidase. In some animals, the oxidation of xanthine to uric acid, a process of purine catabolism, is catalyzed by xanthine oxidase, a molybdenum-containing enzyme. The activity of xanthine oxidase is directly proportional to the amount of molybdenum in the body. However, an extremely high concentration of molybdenum reverses the trend, and can act as an inhibitor in both purine catabolism and other processes. Molybdenum concentrations also affect protein synthesis, metabolism, and growth. These enzymes in plants and animals catalyse the reaction of oxygen in small molecules, as part of the regulation of nitrogen-, sulfur- and carbon cycles.

The average daily intake of molybdenum is believed to be approximately 0.3 mg. Acute toxicity hasn't been seen in humans, and the toxicity depends strongly on the chemical state.

Morus albus

The White Mulberry (Morus alba) is a short-lived, fast-growing, small to medium sized mulberry tree, which grows to 10-20 meters tall. The species is native to northern China, and is widely cultivated (and even naturalized) elsewhere.

In the text Traditional Chinese Medicine, the fruit is used to treat prematurely grey hair, to “tonify” the blood, and treat constipation and diabetes. The bark is used to treat coughing, wheezing, and edema by promoting urination. It is also used to treat fever, headache, red, dry and sore eyes.

Morus alba is used to reduce blood sugar, blood pressure and blood fat. In addition, it is both antibacterial and antiviral. Additional uses include diuresis and sedation.

MSM (methylsulfonylmethane)

Methylsulfonylmethane (MSM), is a natural sulfur compound found in all living things, first isolated by Robert Herschler and Dr. Stanley Jacob of the University of Oregon Medical School in the early 80's. MSM is an odorless, water-soluble, white crystalline material, that supplies a bioavailable form of dietary sulfur, which plays a major role in stabilizing and promoting numerous body functions. Their research revealed that MSM, is actually one of the most prominent compounds in the body, following water and sodium. An 160-pound man contains approximately four pounds of sulfur. Scientists almost missed MSM when studying nutritional needs because it plays such a small part in the food chain. However in his research, Dr. Jacob determined that the sulfur in MSM, called sulfonyl, is as safe and as important as vitamin C in the diet, unlike other forms of sulfurs such as: sulfas, sulfates, sulfites and sulfides that are bad for the body.

MSM originates in the ocean and reaches the human food chain through rainfall. It is the prime source of bio-available sulfur, which is typically removed from food by processing, drying, cooking and preserving. MSM is an important nutrient (not a drug or medicine) and is a component of over 150 compounds. It is needed by the body for healthy connective tissues and joint function, proper enzyme activity and hormone balance, along with proper function of the immune system. Because bio-nutritional sulfur plays such a major role in these healthy body functions as well as others, it was found that supplementation with MSM improves many health problems such as: allergies, asthma, emphysema, lung dysfunction, arthritis, headaches, skin problems, stomach and digestive tract problems, circulation, cell osmosis and absorption. MSM acts as an analgesic and anti-inflammatory. It also inhibits muscle spasm and increases blood flow. MSM is critical to the formation of connective tissue of all types. Without sufficient MSM sulfur in the body, unnecessary illnesses of varying types may result. Research has shown that MSM is helpful in improving joint flexibility, reducing stiffness and swelling, improving circulation and cell vitality, reducing pain and scar tissue, and in breaking up calcium deposits. MSM is responsible for the flexible bond between cells, including those that make up the skin. It acts to block undesired chemical and physical cross-linking or bonding of collagen, which is associated with tough, aging skin. Approximately half of the total body sulfur is concentrated in the body's muscles, skin and bones. It is present in keratin, the tough substance in the skin, nails and hair. Sulfur is necessary for making collagen, the primary constituent of cartilage and connective tissue, but it is also responsible for the conformation of body proteins through the formation of disulfide bonds, which hold connective tissue together. MSM does this in conjunction with vitamins and amino acids. The body never stops making new cells, and this calls for an unceasing supply of MSM. Tests conducted with laboratory animals indicated that wound healing occurred faster in a group receiving MSM, but even faster with both MSM and vitamin C supplementation.

Mucuna pruriens

Mucuna pruriens (syn. Dolichos pruriens) is a tropical legume known by a multitude of common names including Velvet bean, Cowitch, Cowhage, Juckbohne (German: “Itch bean”), Picapica, Kapikachu, Yerepe (Yoruba), and Atmagupta.

In history, Mucuna pruriens has been used as an aphrodisiac. It is still used to increase libido in both men and women due to its dopamine inducing properties. Dopamine has a profound influence on sexual function. A typical dose for a man is 15 grams of ground seeds mixed with cow's milk.

Mucuna pruriens seeds have also been found to have antidepressant properties when consumed. Dried leaves of Mucuna pruriens are sometimes smoked.

Mucuna pruriens seeds contain high concentrations of levodopa, a direct precursor of the neurotransmitter dopamine.

It has long been used in traditional Ayurvedic Indian medicine for diseases including Parkinson's disease. In large amounts (e.g. 30 gram dose) it has been shown to be as effective as pure levodopa/carbidopa in the treatment of Parkinson's disease, but no data on long-term efficacy and tolerability is available.

In addition to levodopa, Mucuna pruriens also contains 5-HTP, nicotine, N,N-DMT, bufotenine, and 5-MeO-DMT. As such, it could potentially have psychedelic effects, and it has purportedly been used in ayahuasca preparations.

The mature seeds of the plant contain about 3.1-6.1% L-DOPA, also trace amounts of bufotenine, DMT, DMT-n-oxide, 5-MeO-DMT-n-oxide, beta-carboline, nicotine and 5-hydroxytryptamine.

The leaves contain about 0.5% L-DOPA, 0.006% dimethyltryptamine (DMT), 0.0025% 5-MeO-DMT and 0.003% DMT n-oxide.

Muira puama

Muira puama is a small Brazilian tree that grows across the Amazon River basin. It has a long history of use in Brazilian folk medicine as an aphrodisiac. Other names include Potency Wood, Ptychopetalum olacoides, and Ptychopetalum uncinatum. The root and stem of the tree are used medicinally. Muira puama is used mainly as an herbal remedy for erectile dysfunction and sexual dysfunction in women. Preliminary animal studies suggest that Muira puama is also a mild stimulant.

Nettle Root Extract (Urticaceae urtica)

Nettle, or stinging nettle, is a perennial plant growing in temperate and tropical wasteland areas around the world.

In folk medicine nettle plants have been used as a diuretic, to build the blood, and as a treatment for arthritis and rheumatism. Externally it has been used to improve the appearance of the hair, and is said to be a remedy against oily hair and dandruff. Nettle root is an effective adjunct to Saw Palmetto for the treatment of Enlarged Prostate.

Nettle inhibits the Aromatase and 5-Alpha Reductase enzymes that have been implicated in the development of Enlarged Prostate. Nettle also inhibits the transformation of the benign cells involved in enlarged prostate to the malignant Cells involved in Prostate Cancer. Nettle inhibits the binding of Dihydrotestosterone (DHT) to Prostate cells (thereby preventing DHT from stimulating the proliferation of prostate cells that leads to Enlarged Prostate, a key underlying factor in the progression of Enlarged Prostate).

Nettle inhibits the binding of Testosterone to Sex Hormone Binding Globulin (SHBG), resulting in lower levels of “bound” Testosterone and higher levels of “free” Testosterone (this effect occurs as a result of Nettle binding to SHBG in place of Testosterone).

### Olive Leaf Extract

The Bible refers to the olive tree as the “tree of life”. Olives, the ripe fruit of the tree, yield healthy monounsaturated fats and phytochemicals that act as potent antioxidants. Research on the benefits of olive oil abounds in scientific literature, yet, people are generally still not aware of the amazing healing powers of another component of the olive tree, the leaves.

The Olive leaf appears to be beneficial in treatment for conditions caused by, or associated with, a virus, retrovirus, bacterium or protozoan. Among those treatable conditions are: influenza, the common cold, candida infections, meningitis, Epstein Barr virus (EBV), encephalitis, herpes I and II, human herpes virus 6 and 7, shingles (Herpes zoster), HIV/ARC/AIDS, chronic fatigue, hepatitis B, pneumonia, tuberculosis, gonorrhea, malaria, dengue, severe diarrhea, and dental, ear, urinary tract and surgical infections.

Many people who live stressful lives, or who may be particularly susceptible to colds and viruses, may benefit from long-term use of olive leaf as a preventive agent. Some patients have expressed other unexpected benefits of olive leaf, including improved psoriasis, normalization of heart beat irregularities, diminished cravings, less pain from hemorrhoids, toothaches and chronically achy joints.

Researchers in Europe investigated the properties of oleuropein and suggested that it inactivates bacteria by dissolving the outer lining of the microbes. More recent studies of oleuropein show that it seems to protect low-density lipoprotein from oxidation, thus reducing the LDL (bad cholesterol). Another research study in France remarks that Olive leaf extracts show extremely high antioxidant qualities.

Olive leaf extract seems to show itself as an immune system builder by directly stimulating phagocyte production (phogocytosis). It also contains natural flavinoids and esters that create a structural complex that infectious microorganisms may not readily develop a resistance to. Biochemists have determined that the extract has multiple iridoids and that the synergy of them working together is the reason for the wide spectrum effect.

Omega-3 (Alpha-Linolenic Acid from Flaxseed

Flaxseed oil is known to be a source of alpha-linolenic acid, an omega-3 fatty acid that can be converted to another omega-3 fatty acid called eicosapentaenoic acid (EPA) and that may reduce the risk of death from heart disease. Questions have been raised about how efficiently this conversion occurs and whether people who do not eat fish (a good source of EPA) can rely on flaxseed oil and other sources of alpha-linolenic acid being converted to EPA.

Omega-3 (Docosahexaenoic Acid from Flaxseed)

Docosahexaenoic acid (commonly known as DHA; 22:6(ω-3), all-cis-docosa-4,7,10,13,16,19-hexa-enoic acid; trivial name cervonic acid) is an omega-3 essential fatty acid. In chemical structure, DHA is a carboxylic acid with a 22-carbon chain[1] and six cis double bonds; the first double bond is located at the third carbon from the omega end.

DHA deficiency is associated with cognitive decline. PS controls apoptosis, and low DHA levels, lower neural cell PS and increase neural cell death. DHA is depleted in the cerebral cortex of severely depressed patients.

A large drug trial is currently recruiting patients for evaluating DHA in Alzheimer's disease. This is the first human trial of DHA. Animal studies in the TG3 transgenic mouse model of Alzheimer's disease linked decreases in amyloid plaques and tau to dietary DHA. Animal studies also show that when combined with arachidonic acid (also present in fish oil), the effectiveness of DHA for preventing plaques was less than without it.

Omega-3 (Eicosapentoenoic Acid from Flaxseed)

Eicosapentaenoic acid (EPA or also icosapentaenoic acid) is an omega-3 fatty acid. In physiological literature, it is given the name 20:5(n-3). It also has the trivial name timnodonic acid. In chemical structure, EPA is a carboxylic acid with a 20-carbon chain and five cis double bonds; the first double bond is located at the third carbon from the omega end.

EPA and its metabolites act in the body largely by their interactions with the metabolites of arachidonic acid.

EPA is a polyunsaturated fatty acid that acts as a precursor for prostaglandin-3 (which inhibits platelet aggregation), thromboxane-3, and leukotriene-5 groups (all eicosanoids).

The US National Institute of Health's Medline Plus lists a large number of conditions in which EPA (alone or in concert with other ω-3 sources) is known or thought to be effective. Most of these involve its ability to lower inflammation.

Among omega-3 fatty acids, in particular EPA is thought to possess beneficial potential in mental conditions, such as schizophrenia. Several studies report an additional reduction in scores on symptom scales used to assess the severity of symptoms, when additional EPA is taken.

Recent studies have suggested that EPA may affect depression, and importantly, suicidal behavior. One such study, took blood samples of 100 suicide-attempt patients and compared the blood samples to those of controls and found that levels of Eicosapentaenoic acid were significantly lower in the washed red blood cells of the suicide-attempt patients.

EPA has inhibitory effect on CYP2C9 and CYP2C19 hepatic enzymes. At high dose, it may also inhibit the activity of CYP2D6 and CYP3A4, important enzymes involved in drug metabolism.

### Omega 6 (Gamma-Linolenic Acid)

Gamma-linolenic acid (GLA) is an essential fatty acid (EFA) in the omega-6 family that is found primarily in plant-based oils. EFAs are essential to human health but cannot be made in the body. For this reason, they must be obtained from food. EFAs are needed for normal brain function, growth and development, bone health, stimulation of skin and hair growth, regulation of metabolism, and maintenance of reproductive processes.

### Omega 6 (Linoleic Acid)

Linoleic acid (LA), another omega-6 fatty acid, is found in cooking oils and processed foods and converted to GLA in the body. GLA is then broken down to arachidonic acid (AA) and/or another substance called dihomogamma-liolenic acid (DGLA). LA can also be consumed directly from meat, and GLA is available directly from evening primrose oil (EPO), black currant seed oil, and borage oil. Most of these oils also contain some linoleic acid.

### Omega 9 (Oleic Acid)

n-9 fatty acids (popularly referred to as ω-9 fatty acids or omega-9 fatty acids) are a family of unsaturated fatty acids which have in common a carbon-carbon double bond in the n-9 position; that is, the ninth bond from the end of the fatty acid.

Omega 9 is a monounsaturated fatty acid present in all animal and vegetable fats. Oleic acid is the most common Omega 9 fatty acid. This fatty acid is essential, but it is not considered an EFA (essential fatty acid) because the human body can manufacture a limited amount of it if other EFA's are present. Omega 9 is found in olive, canola, and grapeseed, peanut, safflower and sunflower oils; however, other sources are avocados, peanuts, peanut butter, nuts and seeds.

Monosaturated Oleic Acid lowers heart attack risk, arteriosclerosis, and can aid in cancer prevention. Omega 9 also naturally lowers cholesterol.

### Ornithine

Ornithine is an amino acid that plays a role in the urea cycle. Ornithine is one of the products of the action of the enzyme arginase on L-arginine, creating urea. Therefore, ornithine is a central part of the urea cycle, which allows for the disposal of excess nitrogen.

Ornithine is not an amino acid coded for by DNA, and, in that sense, is not involved in protein synthesis. However, in mammalian non-hepatic tissues, the main use of the urea cycle is in arginine biosynthesis, so as an intermediate in the metabolic processes, L-ornithine is quite important.

### Ox Bile

Bile is a secretion of liver, stored in the gall bladder until needed. Bile has a major role to play in fat digestion and absorption. The active constituents of bile are the bile salts, which are either glycine or taurine conjugates of polyhydroxy steroidal acid. Since dietary fat cannot be dissolved in water, a special type of system is designed in our body for its digestion and its absorption through the intestinal wall by bile salts. Bile salts are strongly amphilic and with the aid of biliary phospholipids, these are readily fonn micelles and emulsify lipids. These are important not only for emulsification of cholesterol and other lipids on food but also for the emulsfication of dietary lipids. Bile also stimulates the secretion of an enzyme responsible for the breakdown of fats. Bile aids in the production of an alkaline reaction in the intestines and is essential for absorption of fats. Bile emulsifies the fats permitting intestinal and pancreatic lipases to split the triglycerides into diglycerides and monoglycerides and finally into free fatty acids and glycerol.

Cholerectic action of bile extract is attributed to bile acids. Bile Extract also has an important role to play in the absorption of fat-soluble vitamins A, D, E and K. As a constituent of intestinal contents, Ox bile acts as an emulsifying agent and inhibits certain putrefactive bacteria.

### Pancreatin

Pancreatin is a mixture of several digestive enzymes produced by the exocrine cells of the pancreas. It is composed of amylase, lipase and protease. This mixture is used to treat conditions in which pancreatic secretions are deficient, such as pancreatitis and cystic fibrosis. It has been claimed to help with food allergies, celiac disease, autoimmune disease, cancer, and weight loss. Pancreatin is sometimes called “pancreatic acid”, although it is neither a single chemical substance nor an acid.

Pancreatin contains the pancreatic enzymes trypsin, amylase, and lipase. A similar mixture of enzymes is sold as pancrelipase, which contains more active lipase enzyme than does pancreatin. The trypsin found in pancreatin works to hydrolyze proteins into oligopeptides; amylase hydrolyzes starches into oligosaccharides and the disaccharide maltose; and lipase hydrolyzes triglycerides into fatty acids and glycerols. Pancreatin is an effective enzyme supplement for replacing missing pancreatic enzymes, and aids in the digestion of foods in cases of pancreatic insufficiency.

### Pantothenic Acid

Pantothenic acid, also called vitamin B5, is a water-soluble vitamin required to sustain life. Pantothenic acid is needed to form coenzyme-A (CoA), and is critical in the metabolism and synthesis of carbohydrates, proteins, and fats. In chemical structure, it is the amide between D-pantoate and beta-alanine. Its name is derived from the Greek pantothen (παντóθεν) meaning “from everywhere” and small quantities of pantothenic acid are found in nearly every food, with high amounts in whole-grain cereals, legumes, eggs, meat, and royal jelly. It is commonly found as its alcohol analog, the provitamin panthenol, and as calcium pantothenate.

The derivative of pantothenic acid, pantothenol, is a more stable form of the vitamin and is often used as a source of the vitamin in multivitamin supplements. Another common supplemental form of the vitamin is calcium pantothenate. Calcium pantothenate is often used in dietary supplements because as a salt, it is more stable than pantothenic acid in the digestive tract allowing for better absorption.

Doses of 500-1200 mg/day of pantothine has been shown to reduce total serum cholesterol, LDL-cholesterol, and triglycerides, and it may increase HDL-cholesterol. Doses of 2 g/day of calcium pantothenate may reduce the duration of morning stiffness, degree of disability, and pain severity in rheumatoid arthritis patients. Although the results are inconsistent, supplementation may improve oxygen utilization efficiency and reduce lactic acid accumulation in athletes.

Pantothenic acid deficiency is exceptionally rare and has not been thoroughly studied. In the few cases where deficiency has been seen (victims of starvation and limited volunteer trials), nearly all symptoms can be reversed with the return of pantothenic acid.

Symptoms of deficiency are similar to other vitamin B deficiencies. Most are minor, including fatigue, allergies, nausea, and abdominal pain. In a few rare circumstances more serious (but reversible) conditions have been seen, such as adrenal insufficiency and hepatic encephalopathy.

It has been noted that painful burning sensations of the feet were reported in tests conducted on volunteers. Deficiency of pantothenic acid may explain similar sensations reported in malnourished prisoners of war.

Deficiency symptoms in other non-ruminant animals include disorders of the nervous, gastrointestinal, and immune systems, reduced growth rate, decreased food intake, skin lesions and changes in hair coat, alterations in lipid and carbohydrate metabolism.

### Papain

Papain is a cysteine protease (EC 3.4.22.2) hydrolase enzyme present in papaya (Carica papaya) and mountain papaya (Vasconcellea cundinamarcensis). It consists of 212 amino acids stabilized by 3 disulfide bridges. Its 3D structure consists of 2 distinct structural domains with a cleft between them. This cleft contains the active site, which contains a catalytic triad that has been likened to that of chymotrypsin. Its catalytic triad is made up of 3 amino acids—cysteine-25 (from which it gets its classification), histidine-159, and asparagine-158.

The mechanism by which it breaks peptide bonds involves deprotonation of Cys-25 by His-159. Asp-158 helps to orient the imidazole ring of His-159 to allow this deprotonation to take place. Cys-25 then performs a nucleophilic attack on the carbonyl carbon of a peptide backbone. This frees the amino terminal of the peptide, and forms a covalent acyl-enzyme intermediate. The enzyme is then deacylated by a water molecule, and releases the carboxy terminal portion of the peptide. In immunology, papain is known to cleave the Fc (crystallisable) portion of immunoglobulins (antibodies) from the Fab (antigen-binding) portion.

Papain is used to dissociate cells in the first step of cell culture preparations. A 10-minute treatment of small tissue pieces (less than 1 mm cubed) will allow papain to begin breaking down the extracellular matrix molecules holding the cells together. After 10 minutes, the tissue should be treated with a protease inhibitor solution to stop the protease action (if left untreated papain's activity will lead to complete lysis of the cells). The tissue must then be triturated (passed quickly up and down through a Pasteur pipette) in order to break up the pieces of tissue into a single cell suspension.

It is also used as an ingredient in various enzymatic debriding preparations, notably Accuzyme®. These are used in the care of some chronic wounds to clean up dead tissue.

### Pepsin

Pepsin is a digestive protease produced in the mucosal lining of the stomach that acts to degrade protein. Pepsin is one of three principal protein-degrading, or proteolytic, enzymes in the digestive system, the other two being chymotrypsin and trypsin. During the process of digestion, these enzymes, each of which is particularly effective in severing links between particular types of amino acids, collaborate to break down dietary proteins to their components, i.e., peptides and amino acids, which can be readily absorbed by the intestinal lining. In the laboratory studies pepsin is most efficient in cleaving bonds involving the aromatic amino acids, phenylalanine, tryptophan, and tyrosine. Pepsin is synthesized in an inactive form by the stomach lining; hydrochloric acid, also produced by the gastric mucosa, is necessary to convert the inactive enzyme and to maintain the optimum acidity (pH 1-3) for pepsin function. Pepsin and other proteolytic enzymes are used in the laboratory analysis of various proteins; pepsin is also used in the preparation of cheese and other protein-containing foods.

Pfaffia paniculta (Suma)

Suma also called Brazilian ginseng (Pfaffia paniculata syn. Hebanthe paniculata, Gomphrena paniculata, Gomphrena eriantha, Iresine erianthos, Iresine paniculata, Iresine tenuis, Pfaffia eriantha, Xeraea paniculata. Pfaffia paniculta is the root of a rambling ground vine found in South America used traditionally as a medicine and tonic. Nicknamed “para todo” which means “for all,” suma is an herbal medicine with adaptogenic qualities that serve to normalize and enhance body systems, increase resistance to stress, and boost overall functioning. It has been used for a variety of ailments with good efficacy, hence the name “para todo.”

Suma is said to support hormonal balance, reduce inflammation, inhibit cancer and leukemia cells, enhance immunity, increase libido, and provide a number of normalizing and rejuvenating effects. One of the reasons for its myriad effects may be its ability to increase oxygenation and energy efficiency at the cellular level. Suma contains germanium, beta-ecdysterone, allantoin, and a group of novel phytochemical saponins called pfaffosides.

Called the “Russian Secret”, suma root has long been used by athletes from that part of the world as an alternative to steroids. This may be because it contains the phytochemical beta-ecdysterone, and several related chemicals, as well as an unusual concentration of common nutrients and amino acids.

Phosphatidylserine (from Soy Lecithin)

Phosphatidylserine (abbreviated Ptd-L-Ser, or PS) is a phospholipid component, usually kept on the inner-leaflet, the cytosolic side, of cell membranes by an enzyme called translocase. Phosphatidylserine (PS) is classified as a phospholipid, that is, a fat that is found in every cell of the human body. Phosphatidylserine can be found in very high concentrations in the brain, where it is responsible for keeping cell membranes fluid, flexible, and ready to process essential nutrients.

On May 13, 2003, the U.S. Food and Drug Administration stated “based on its evaluation of the totality of the publicly available scientific evidence, the agency concludes that there is not significant scientific agreement among qualified experts that a relationship exists between phosphatidylserine and reduced risk of dementia or cognitive dysfunction.” The FDA did, however give “qualified health claim” status to phosphatidylserine, stating that “Consumption of phosphatidylserine may reduce the risk of dementia in the elderly” and “Consumption of phosphatidylserine may reduce the risk of cognitive dysfunction in the elderly”.

Phosphatidylserine has been demonstrated to speed up recovery, prevent muscle soreness, improve well-being, and might possess ergogenic properties in athletes involved in cycling, weight training and endurance running. PS has been reported to be an effective supplement for combating exercise-induced stress by blunting the exercise-induced increase in cortisol levels. PS supplementation promotes a desirable hormonal balance for athletes and might attenuate the physiological deterioration that accompanies overtraining and/or overstretching. In recent studies, PS has been shown to enhance mood in a cohort of young people during mental stress and to improve accuracy during tee-off by increasing the stress resistance of golfers.

First pilot studies indicate that PS supplementation might be beneficial for children with attention-deficit hyperactivity disorder.

PS is also a very important support chemical for nerve tissue. As a support to the nervous system, PS aids proper release and reception of neurotransmitters in the brain. PS helps to ensure that memory-related pathways function smoothly.

Some research shows that when PS levels naturally decrease with increasing age, so too does the ability to learn, remember, and stay alert. Depression may also develop as a result of age-related phosphatidylserine insufficiency. PS supplements have been proposed as a partial solution to such developments. Rapidly absorbed into the brain, these supplements may compensate for low PS levels and thus prevent or even reverse age-related declines in brain function. PS actually appears in numerous foods, including rice and green leafy vegetables, but only in small amounts. Commercially prepared supplements offer a concentrated source. Health Benefits and Indications Studies indicate that phosphatidylserine supplements may help to revitalize age-impaired brain function. However, there is no solid research evidence to indicate that adding PS supplements to the diet in this way will make any difference. Specifically, phosphatidylserine may help to lessen age-related decline in brain functioning, slow brain function decline in Alzheimer's disease and Parkinson's disease, combat depression, and increase the ability to handle stress.

### Phosphorus

Phosphorus is an essential mineral that is required by every cell in the body for normal function. The majority of the phosphorus in the body is found as phosphate (PO₄). Approximately 85% of the body's phosphorus is found in bone.

Phosphorus is a major structural component of bone in the form of a calcium phosphate salt called hydroxyapatite. Phosphorus s also necessary for the formation of the phospholipids: lecithin and cephalin, which are integral parts of cell structures. Phospholipids (e.g., phosphatidylcholine) are major structural components of cell membranes. All energy production and storage are dependent on phosphorylated compounds, such as adenosine triphosphate (ATP) and creatine phosphate. Nucleic acids (DNA and RNA), which are responsible for the storage and transmission of genetic information, are long chains of phosphate-containing molecules. A number of enzymes, hormones, and cell-signaling molecules depend on phosphorylation for their activation. Phosphorus also helps to maintain normal acid-base balance (pH) by acting as one of the body's most important buffers. Additionally, the phosphorus-containing molecule 2,3-diphosphoglycerate (2,3-DPG) binds to hemoglobin in red blood cells and affects oxygen delivery to the tissues of the body.

Phosphorus is essential for the proper utilization of not only calcium but also other minerals like iron, magnesium, potassium, and sodium. In combination with calcium, it feeds the nerves.

Phosphorus is indispensable for all active tissues. It aids the growth of hair and helps counteract fatigue. This mineral is important for the regular functioning of the heart and for normal kidney functioning. Phosphorus is essential in the chemical processes involved in the utilization of carbohydrates and fats. It liberates their energy at the rate demanded by the body.

### Potassium

Potassium is an element (and an electrolyte) that's essential for the body's growth and maintenance. While necessary to keep a normal water balance between the cells and body fluids, Potassium also plays an essential role in the response of nerves to stimulation and in the contraction of muscles. Cellular enzymes need potassium to work properly.

A potassium deficiency due to increased urinary loss often occurs when medication for certain heart diseases is used to prevent sodium and water retention. To overcome this loss, physicians often suggest eating more foods high in potassium. More potassium may be prescribed as a medicine.

Foods high in potassium include bananas, cantaloupe, grapefruit, oranges, tomato or prune juice, honeydew melons, prunes, molasses and potatoes. Some foods high in potassium are also high in calories. When weight control is important, eat more low-calorie foods. Foods such as fruits, vegetables and low-fat dairy products that are high in potassium and calcium, when incorporated in the DASH Diet Study, helped to significantly lower blood pressure.

Orally, potassium is used for treating and preventing hypokalemia, hypertension, Meniere's disease, thallium poisoning, hypercalciuria, insulin resistance, myocardial infarction, stroke prevention, symptoms of menopause, and infant colic. It is also used orally for allergies, headaches, acne, alcoholism, Alzheimer's disease, arthritis, blurred vision, cancer, chronic fatigue syndrome, colitis, confusion, constipation, dermatitis, edema, fever, gout, insomnia, irritability, mononucleosis, muscle weakness, muscular dystrophy, stress, and as an adjunct for treating myasthenia gravis.

### Polygonum Multiflorum

Polygonum multiflorum, better known as Chinese knotweed or Flowery knotweed, is a widely used Chinese herb. It is said to rejuvenate the body, and is also commonly known as fo-ti, fo-ti-teng, ho shou wu, or he shou wu. He Shouwu, a Chinese man who is reputed to have lived until the age of 132, is the first consumer of this herb to be reported.

The Chinese patent medicine called Shou Wu Chih has Polygonum multiflorum as one of its primary ingredients.

In Traditional Chinese Medicine (TCM), Polygonum multiflorum is believed to tonify the kidneys, balance a fragile yin, treat weak bones and premature graying of hair, and protect the skin against UVB damage.

When taken internally, Polygonum multiflorum also has a laxative effect.

Polygonum multiflorum contains stilbene glycosides similar to resveratrol and with superior antioxidant activity. Resveratrol is extracted from Polygonum multiflorum's close relative, Japanese knotweed (P. cuspidatum). Resveratrol has been suggested to extend lifespan by a variety of mechanisms.

### Protease

A protease is any enzyme that conducts proteolysis, that is, begins protein catabolism by hydrolysis of the peptide bonds that link amino acids together in the polypeptide chain. Proteolytic enzymes are very important in digestion as they breakdown the peptide bounds in the protein foods to liberate the amino acids needed by the body. Additionally proteolytic enzymes have been used for a long time in various forms of therapy. Their use in medicine is notable based on several clinical studies indicating their benefits in oncology, inflammatory conditions, and immune regulation. Protease is able to hydrolyze almost all proteins as long as there are not components of living cells. Normal living cells are protected against lysis by the inhibitor mechanism. Parasites, fungal forms, and bacteria or protein. Viruses are cell parasites consisting of nucleic acids covered by a protein film. Enzymes can break down undigested protein cellular debris, and toxins in the blood, sparing the immune system this task. The immune system can then concentrate it full action on the bacterial or parasitic invasion.

### Pumpkin Seed Extract

Pumpkin seeds are one of nature's almost perfect foods. They are a natural source of beneficial constituents such as carbohydrates, amino acids and unsaturated fatty acids. They contain most of the B vitamins, along with vitamins C, D, E, and K. They also have the minerals calcium, potassium, niacin, and phosphorous. Pumpkin seeds have mainly been used to treat prostate and bladder problems, but they have also been known to help with depression and learning disabilities. Native American tribes were among the first people to notice the beneficial aspects of pumpkin seeds. They referred to them as cucurbita and used them to treat kidney problems and to eliminate parasites from the intestines.

Because pumpkin seeds turned up so frequently in folk medicines, scientists began to conduct research on the oil extracted from them. One study showed pumpkin seed oil kept hormones from inflicting damage on the cells of the prostate, which helps to reduce cancer development. Another study revealed that the seeds contain a significant amount of L-tryptophan, which is beneficial in battling depression (although it is believed the seeds don't have enough to treat major depression, they can be used as a preventive measure). Other studies showed pumpkin seeds can improve bladder and urethra function, and they are thought to help stop the formation of kidney stones. Pumpkin seeds have also been used to treat learning disorders and are generally recommended in some countries as a “brain food.” Other studies have shown they prevent hardening of the arteries and help regulate cholesterol levels.

Pumpkin seeds promote overall prostate health and alleviate the difficult urination associated with an enlarged prostate. In some studies, pumpkin seed extracts improved bladder function in animals.

Pumpkin seeds are high in zinc; pumpkin seeds are a natural protector against osteoporosis. Low intake of zinc is linked to higher rates of osteoporosis.

Pumpkin seeds effectively reduce inflammation without the side effects of anti-inflammatory drugs, prevent kidney stones Pumpkin.

Pumpkin seeds are used in many cultures as a natural treatment for tapeworms and other parasites. Studies also show them to be effective against acute schistosomiasis, a parasite contracted from snails.

Pumpkin seeds contain phytosterols, compounds that that have been shown to reduce levels of LDL cholesterol.

Rhodiola rosea

A substantial proportion of the diseases and illnesses which accrue as a person ages is considered to be caused by years of living with elevated stress levels. One of the best stress reducing remedies available to fight the ongoing effects of stress filled lives, are herbal products called adaptogens, which possess many curative properties. One such adaptogen is Rhodiola rosea. Amongst the many benefits attributed to it are improvements in memory, concentration and attention span; reduction of anxiety and panic attacks; the alleviation of depression; and a decrease in mental and physical fatigue. Rhodiola rosea is a plant that is native to the world's colder regions, including the Arctic, Central Asia, the Rocky Mountains of North America, as well as certain mountainous locales in Western Europe. Rhodiola rosea, as a stress-retarding adaptogen, has the potential to enhance brain function and mood. Stress interferes with memory functions and, over time, causes deterioration in memory systems. In addition to enhancing cognitive functions, learning and memory, Rhodiola rosea may exert positive effects on memory and cognition by improving resistance to physical and emotional stress. Thus, the dual action of cognitive stimulation and emotional calming creates benefits for both immediate cognitive and memory performance and for the long-term preservation of brain functions. And as an antioxidant, Rhodiola rosea may help protect the nervous system from oxidative damage caused by free radicals

The systematic study of the pharmacological effects of Rhodiola rosea began in 1965. Since then, various studies, predominantly in Russia (where it has for decades been taken by the military, cosmonauts and athletes as an effective anti-aging remedy) and in Germany, show that it can be used in a number of beneficial ways such as a stimulant for the symptoms of asthenia (decline in work performance, sleep difficulties, poor appetite, irritability, headaches, and fatigue). In healthy individuals it may be used to relieve fatigue, increase attention span, memory and work productivity. Overall, in small and medium doses, Rhodiola rosea enhances norepinephrine, dopamine, serotonin, and nicotinic cholinergic effects in the central nervous system. Rhodiola rosea fights the physical and mental effects of stress and lowers cortisol levels. It also promotes the synthesis of ATP (cellular fuel) so as to increase physical work capacity and dramatically shorten the recovery time between bouts of high-intensity exercise. Rhodiola rosea has the potential to help patients with depressive syndromes, mental and physical fatigue, memory loss and cognitive dysfunction from a variety of causes. It helps with sexual dysfunction, and menopausal-related disorders. Rhodiola rosea is the premium choice to counteract the mental and physical effects of stress. It is a potent herb that helps reduce levels of the stress hormone cortisol while enhancing levels of key brain chemicals involved in regulating mood. In addition, Rhodiola rosea works naturally to promote the synthesis of ATP, the primary energy source for all cells in the body.

Rhododendron caucasicum

Rhododendron caucasicum or Siberian Rhododendron caucasicum (S.R.C.), common name: Georgian Snow Rose, is an elegant evergreen shrub inhabiting the mountainous regions of the Caucasian mountains of Russia at elevations of 10,000 to 13,000 feet. It has a history of regional usage spanning over 400 years. S.R.C. contains a compound that is unique for “fat control” and antioxidant properties. However, the benefits of this wondrous plant, reach far beyond its effective natural weight management benefits to also being used in the treatment of chronic illnesses such as rheumatism, arthritis, high blood pressure and gout. Other S.R.C. physiological benefits include stimulation of body heat and an increase of diaphoresis; which is the secretion, then elimination of toxins and waste from the body.

Healthy bodies need certain levels of fat in order to perform properly. These fats, called essential fatty acids (omega 3 & omega 6 fatty acids) when in balance, provide necessary nutrients for the brain, hair, nails, skin, muscles, nerves and include a long list of other essential metabolic benefits. Therefore it is not wise to cut out all fat from the diet, but balance fat by blocking excess amounts. S.R.C. acts in the same way as pharmaceutical drugs (fat blockers/lipase inhibitors) in that it safely and selectively blocks lipase activity. Unlike drugs, the clinical trials of S.R.C. have shown that it does not reduce lipase function to a level that can threaten normal metabolic balance, yet safely blocks enough fat to make a difference without the risk of compromising your health. This occurs because the phenolics of S.R.C. specifically inhibit the activity of the lipase key enzyme in fat metabolism, but does not inhibit the effects of the other enzymes necessary in digestion such as trypsin, pepsin and amylase. The studies show that using S.R.C. three times a day, before meals produced a natural 15 20% decrease in fat assimilation. These tests clearly indicated that S.R.C. is a safe and promising natural alternative to pharmaceutical drugs; which may cause harmful side effects.

In the Republic of Georgia, it is not unusual for people to live beyond 100 years of age as active members of society. Even today Siberian Rhododendron caucasicum remains a very popular traditional drink in the Republic of Georgia and is revered as one of the secrets to Georgian longevity. When Russians marry they propose a toast to the bride and groom gifting them Georgian longevity. What is their secret? For over 2,000 years, Georgians have consumed Alpine Tea as a daily ritual along with grain kefir containing eleven different probiotics. In addition, the water they drink is full of glacial minerals. It has been theorized that their long lives may be attributed to regular consumption of this traditional yogurt (kefir) and flavonoid rich foods such as wine and honey, along with their Alpine Tea.

The last verifiable statistics from the Republic of Georgia show that there are almost 23,000 Georgians over the age of 100 based on a population of only 3.2 million people. One of the key reasons for their longevity is Rhododendron caucasicum, the ingredient in Alpine Tea. Grown at 10,000 to 30,000 foot elevations in the Caucasian Mountains, Rhododendron caucasicum contains polyphenolics, including flavonoids and proanthocyanidins. Thirty years of research indicate that the phenylpropanoids in Rhododendron caucasicum improve physical abilities, increase activity of the cardiovascular system, and increase blood supply to the muscles and especially to the brain. Rhododendron caucasicum increases resistance of the brain to imbalances due to chemical, physical, and biological reasons. It also is an antibacterial while allowing the good probiotics to thrive. It acts as a detoxicant, is highly P vitamin active, protecting against capillary fragility, and is an excellent free radical scavenger. Studies have demonstrated that Rhododendron caucasicum inhibits or abolishes the activity of the enzyme hyaluronidase, known to be an initiator of colon cancer.

Clinical research has been ongoing as to the medicinal uses of this alpine plant. Prof. Dimitry M. Rossiyski, M.D., Meritorious Science Worker at the Soviet Medical Academy of Science, conducted a double blind placebo study on seventy test subjects diagnosed with circulatory insufficiency and atherosclerosis, some with high blood pressure and evidence of past heart attacks. A 30-mg/day dose of Rhododendron extract over a 15-day period resulted in the subjects experiencing lower blood pressure, improvement in coronary circulation, decrease of serum cholesterol, and elimination of pain in the chest area. Subsequent studies at the First Central Moscow Hospital showed similar results on heart patients suffering from hardening of the arteries. Doctors Avraamova and Galperin performed clinical studies at the Moscow State Hospital on 24 males and 36 females ages 18 65 diagnosed with mitral valve insufficiency (prolapse). Improvements were obvious in the patients taking Rhododendron extract over those who did not receive it. The average hearts beats in those receiving the extract were lowered from 90 to 70 beats per minute and systolic blood pressure was lowered from 177 to 160 mm Hg (Rossiysky 1954).

Again, studies at the First Central Moscow Hospital revealed that when 50 mg of Rhododendron caucasicum diluted in water was given to 170 volunteers suffering from severe gout, the average discharge of uric acid increased 55-60 percent and pain was relieved within a few hours. The Georgian Academy of Sciences gave 50 to 100 mg per day of the Snow Rose to 114 patients hospitalized for depression. The results showed a marked decrease in depressive symptoms in 93 of the patients. Similar results were achieved by the Moscow State Hospital study, indicating improvement of 162 patients with severe depression.

One of the key benefits for anti-aging is Rhododendron caucasicum's inhibition of hyaluronidase activity and its subsequent benefit to osteoarthritis patients. One theory regarding the cause of arthritis is that it may appear because of the abnormal release of the enzyme hyaluronidase from the cartilage cells. This leads to cartilage breakdown and destruction of the joint. Perhaps its inhibition of hyaluronidase activity is the key to arthritis prevention.

Rhododendron caucasicum is also highly antibacterial, perhaps due to the presence of the well known antibacterial compounds chlorogenic and caffeic acids that are known to exist in this plant. Tests conclude that it is more effective than either grape seed or pine bark as an antibacterial proanthocyanidin. In a 24-hour test of 12,000 Staphylococcus aureus bacteria thriving in solution, all were totally eliminated by Rhododendron, but 300 colonies were still surviving in the grape seed petri dish, and 370 in the pine bark solution.

Rhododendron caucasicum is a unique plant among all other species of Rhododendron and is considered safe for human consumption. It is reported that some Rhododendrons, mainly the flowers, contain grayanotoxins, which are not soluble in water and can be harmful. Therefore, do not just go out to your garden and harvest your backyard Rhododendron! The snow rose has been used in foreign hospitals, to treat heart disease, arthritis, out, high cholesterol, blood pressure problems, depression, neuroses and psychoses, and concentration problems. Along with a strong regime of probiotic supplementation and maintaining a healthy mineral balance in the body, taking Rhododendron caucasicum may give the body the tools it needs to achieve the level of longevity enjoyed by the elder Georgians.

### Selenium

Selenium is a trace mineral that is essential to good health but required only in small amounts. Selenium is incorporated into proteins to make selenoproteins, which are important antioxidant enzymes. The antioxidant properties of selenoproteins help prevent cellular damage from free radicals. Other selenoproteins help regulate thyroid function and play a role in the immune system.

Plant foods are the major dietary sources of selenium in most countries throughout the world. The content of selenium in food depends on the selenium content of the soil where plants are grown or animals are raised. For example, researchers know that soils in the high plains of northern Nebraska and the Dakotas have very high levels of selenium. People living in those regions generally have the highest selenium intakes in the United States (U.S.). In the U.S., food distribution patterns across the country help prevent people living in low-selenium geographic areas from having low dietary selenium intakes. Soils in some parts of China and Russia have very low amounts of selenium. Selenium deficiency is often reported in those regions because most food in those areas is grown and eaten locally.

Results of the National Health and Nutrition Examination Survey (NHANES III-1988-94) indicated that diets of most Americans provide recommended amounts of selenium. The INTERMAP study examined nutrient intakes of almost 5,000 middle-aged men and women in four countries in the late 1990s, including the U.S. The primary aim of the study was to evaluate the effect of dietary micronutrients on blood pressure. Each study participant completed four, 24-hour dietary recalls, during which they were asked to record everything consumed (food, beverages, and dietary supplements) over the previous 24 hours. Selenium intake was lowest among residents of China, the country with the highest known rate of selenium deficiency. Mean dietary intake of selenium of U.S. participants was 153 μg for men and 109 μg for women. Both values exceed the recommended selenium intake for adults and are further evidence of adequate selenium intakes in the U.S.

There is evidence that selenium deficiency may contribute to the development of a weakened immune system. There is also evidence that selenium deficiency does not usually cause illness by itself. Rather, it can make the body more susceptible to illnesses caused by other nutritional, biochemical or infectious stresses.

Three specific diseases have been associated with selenium deficiency. First, Keshan Disease, which results in an enlarged heart and poor heart function, occurs in selenium deficient children; Kashin-Beck Disease, which results in osteoarthropathy; and Myxedematous Endemic Cretinism, which results in mental retardation.

Keshan disease was first described in the early 1930s in China, and is still seen in large areas of the Chinese countryside with selenium poor soil. Dietary intake in these areas is less than 19 micrograms per day for men and less than 13 micrograms per day for women, significantly lower than the current RDA for selenium. Researchers believe that selenium deficient people infected with a specific virus are most likely to develop Keshan disease.

Selenium deficiency has also been seen in people who rely on Total Parenteral Nutrition (TPN) as their sole source of nutrition. TPN is a method of feeding nutrients through an intravenous (IV) line to people whose digestive systems do not function. TPN typically uses forms of nutrients that do not require digestion and/or are dissolved in liquid and infused through the IV line. It is important for TPN solutions to provide selenium in order to prevent a deficiency. Physicians can monitor the selenium status of individuals receiving TPN to make sure they are receiving adequate amounts.

Severe gastrointestinal disorders may decrease the absorption of selenium, resulting in selenium depletion or deficiency. Gastrointestinal problems that impair selenium absorption usually affect absorption of other nutrients as well, and require routine monitoring of nutritional status so that appropriate medical and nutritional treatment can be provided.

Tribulus terrestris

Like many weedy species, Tribulus terrestris has many common names. Puncture Vine, Caltrop, Yellow Vine, and Goathead are the most widely used.

Tribulus terrestris has long been a constituent in tonics in Indian Ayurveda practice, where it is known by its Sanskrit name, “gokshura.”

Some have compared the tonic properties of Tribulus terrestris to the effects of ginseng, but these occur due to entirely different mechanisms. It is also claimed that Tribulus terrestris increases testosterone by increasing gonadotropin-releasing hormones with gonadotropic adaptogen compound (GnRH) (containing Tribulus terrestris) which in turn stimulates the production of LH and follicle-stimulating hormone (FSH). Testosterone, besides its role in muscle-building and raising fertility and libido, is known to have a positive effect on bone marrow activity (for red blood cell production) and the immune system.

Animal studies in rats, rabbits and primates have demonstrated that administration of Tribulus terrestris extract can produce statistically significant increases in levels of testosterone, dihydrotestosterone and dehydroepiandrosterone, and produces effects suggestive of aphrodisiac activity. On the other hand, one recent study found that T. terrestris caused no increase in testosterone or LH in young men, and another found that a commercial supplement containing androstenedione and herbal extracts, including T. terrestris, was no more effective at raising testosterone levels than androstenedione alone.

### Vitamin A

This is the vitamin that has been called the “miracle vitamin” for years because of the positive impact it has on enhancing our immunity and growth. Women also love this vitamin since it is believed to be able to slow the aging process by improving the overall skin condition. The benefits of Vitamin A include acting as an anti-oxidant; helping to protect cells against cancer and other diseases; improving vision and preventing night blindness; promoting formation of strong bones; guarding against bacterial, viral, and parasitic infections; guarding against heart disease and stroke; lowering blood cholesterol levels; improving skin conditions like acne or psoriasis; and eliminating wrinkles, reducing fine lines in the skin and fading age spots.

Vitamin A supplements may help children with respiratory problems.

The supplement is also believed to help people suffering from glaucoma and measles.

### Vitamin B12

Vitamin B12 has a long history and a solid reputation as an energy booster, and it is among the most important of all the B complex vitamins. Vitamin B12 is a water-soluble vitamin required for normal cell activity, DNA replication, and the synthesis of the mood influencing substance SAMe. Vitamin B12, also known as cobalamin, works with folic acid to produce healthy red blood cells. Vitamin B12 also keeps the central nervous system healthy by helping to maintain the myelin sheath that insulates nerve fibers from each other. B 2 deficiency can cause a type of anemia marked by fewer but larger red blood cells that's called pernicious anemia. Prolonged B12 deficiency can lead to nerve degeneration and irreversible neurological damage. People with vitamin B12 deficiency show irregular destruction of the myelin sheaths, which eventually causes paralysis and death. Vitamin B12 levels decrease with age and various measures of cognitive impairment are associated with reduced B 12 status.

### Vitamin D

Vitamin D is a group of fat-soluble prohormones, the two major forms of which are vitamin D2 (or ergocalciferol) and vitamin D3 (or cholecalciferol). The term vitamin D also refers to metabolites and other analogues of these substances. Vitamin D3 is produced in skin exposed to sunlight, specifically ultraviolet B radiation.

Vitamin D plays an important role in the maintenance of organ systems. Vitamin D regulates the calcium and phosphorus levels in the blood by promoting their absorption from food in the intestines, and by promoting re-absorption of calcium in the kidneys, which enables normal mineralization of bone and prevents hypocalcemic tetany. It is also needed for bone growth and bone remodeling by osteoblasts and osteoclasts. Absent vitamin K or with drugs (particularly blood thinners) which interfere with Vitamin K metabolism, Vitamin D can promote soft tissue calcification. It inhibits parathyroid hormone secretion from the parathyroid gland. Vitamin D affects the immune system by promoting phagocytosis, anti-tumor activity, and immunomodulatory functions.

Vitamin D regulates the expression of genes associated with cancers and autoimmune disease by controlling the activation of the vitamin D receptor (VDR), a type 1 nuclear receptor and DNA transcription factor.[6] Research has indicated that vitamin D deficiency is linked to colon cancer and more recently, to breast cancer.[7] Conflicting evidence links vitamin D deficiency to other forms of cancer.

Vitamin D deficiency can result from: inadequate intake coupled with inadequate sunlight exposure, disorders that limit its absorption, conditions that impair conversion of vitamin D into active metabolites, such as liver or kidney disorders, or, rarely, by a number of hereditary disorders.

Vitamin D deficiency is known to cause several bone diseases including rickets, a childhood disease characterized by impeded growth, and deformity, of the long bones. The earliest sign of subclinical vitamin D deficiency is Craniotabes, abnormal softening or thinning of the skull, Osteomalacia, a bone-thinning disorder that occurs exclusively in adults and is characterized by proximal muscle weakness and bone fragility; and Osteoporosis, a condition characterized by reduced bone mineral density and increased bone fragility.

Prior to the fortification of milk products with vitamin D, rickets was a major public health problem. In the United States, milk has been fortified with 10 micrograms (400 IU) of vitamin D per quart since the 1930s, leading to a dramatic decline in the number of rickets cases.

Zinc and Iron are often found to be poorly regulated or even deficient in Vitamin D deficiency. Vitamin D enhances the activities of Vitamin A, and Zinc reduces the toxicity of Vitamin A, while Iron reduces the toxicity of Zinc. These four substances are seen as part of the pathology of Alzheimer's, Parkinson's and some peripheral neuropathies including Restless Leg Syndrome.

Vitamin D malnutrition may also be linked to an increased susceptibility to several chronic diseases such as high blood pressure, tuberculosis, cancer, periodontal disease, multiple sclerosis, chronic pain, depression, schizophrenia, seasonal affective disorder, peripheral artery disease and several autoimmune diseases including type 1 diabetes (see role in immunomodulation).

High levels of vitamin D may slow aging and increase life span by preventing the age-related decline in telomere length, according to a recent report. Telomeres are repetitive DNA sequences at the end of Chromosomes that shorten with aging, cell division, and inflammation. When telomeres become too short, the cell can no longer divide, and it becomes senescent or dies. For this reason, scientists seeking to extend life span have long been interested in methods to prevent telomere shortening. Scientist at King's College, London studied more than 2,000 women examining their serum levels of vitamin D and assessing leukocyte telomere length. Women with the highest levels of vitamin D had the longest telomeres, even after adjusting for age differences. Women with the lowest levels of vitamin D had the highest levels of C-reactive protein, a marker for chronic inflammation. Increasing vitamin D levels through appropriate supplementation may therefore have important benefits for slowing aging and prolonging life. Higher vitamin D concentrations are associated with longer (leukocyte telomere length) which underscores the potentially beneficial effects of vitamin D on aging, and age-related diseases.

### Vitamin E

Vitamin E is the collective name for a set of 8 related tocopherols and tocotrienols, which are fat-soluble vitamins with antioxidant properties. Of these, α-tocopherol (also written as alpha-tocopherol) has been most studied as it has the highest bioavailability, with the body preferentially absorbing and using this form.

It has been claimed that α-tocopherol is the most important lipid-soluble antioxidant, and that it protects cell membranes from oxidation by reacting with lipid radicals produced in the lipid peroxidation chain reaction. This would remove the free radical intermediates and prevent the oxidation reaction from continuing. The oxidised α-tocopheroxyl radicals produced in this process may be recycled back to the active reduced form through reduction by other antioxidants, such as ascorbate, retinol or ubiquinol.

The functions of the other forms of vitamin E are less well studied, although γ-tocopherol (also written as gamma-tocopherol) is a nucleophile that may react with electrophilic mutagens, and tocotrienols may have a specialized role in protecting neurons from damage. However, the roles and importance of the various forms of vitamin E are presently unclear, and it has even been suggested that the most important function of vitamin E is as a signaling molecule, and that it has no significant role in antioxidant metabolism.

Most studies about Vitamin E have supplemented only alpha-tocopherol, but doing so leads to reduced serum gamma- and delta-tocopherol concentrations.

These ingredients, when combined in the quantities indicated in Table I for either the “original”, powered (i.e., capsular), or liquid formulations, provide the anti-aging health supplement of the invention. For the liquid formulation, the indicated quantities are combined in approximately 20 oz of water. It will be recognized that minor variation from the indicated quantities may be acceptable. Consequently, the invention is seen to include such variations in addition to the amounts used for purposes of disclosure.

The formulation has been refined over a period of twenty years to maximize efficacy by maximizing synergistic interactions while virtually eliminating any potential negative outcomes because of interactions among the ingredients. While efficacy of the original formulation has proven to be excellent, it is believed that the alternate embodiment entitled “powder” in Table I is also excellent. Even greater balance and synergy is achieved in this alternated embodiment of the formulation of the invention. It should be noted that the delivery system of the “original” formulation is typically in powdered form. It will further be recognized that the “original” formulation could readily be adapted for delivery in a liquid.

It will be further noted that the formulation of the invention may be packaged in a single dose capsule, unlike some formulations of the prior art. It is anticipated that a single capsule constitutes a normal dose of the novel supplement. Multiple doses of the supplement may be taken in the course of a day in accordance with individual preference or need.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A composition in powder form, comprising: Acacia leaf extract, approximately 5 mg; Alfalfa (Medicago sativa), approximately 35 mg; Alpha Lipoic Acid, approximately 10 mg; Amylase, approximately 5 mg; Astragalus membranaceus, approximately 75 mg; Biotin, approximately 1000 mcg; Bromelain (from pure plant enzymes), approximately 5 mg; Calcium, approximately 10 mg; Carnosine, approximately 5 mg; Cellulase (from pure plant enzymes), approximately 5 mg; Chlorella, approximately 5 mg; Chondroitin, approximately 10 mg; Chromium Picolinate, approximately 5 mcg; Chrysin, approximately 15 mg; Lily extract (Clintonia borealis), approximately 5 mg; Copper, approximately 3.5 mcg; Co-Q10, approximately 15 mg; Cyanotis Vaga, approximately 20 mg; Cystoseira canariensis (from Brown Sea Algae), approximately 7.5 mg; DHEA, approximately 3 mg; Emblica officinalis, approximately 25 mg; Ester-C, approximately 10 mg; Fucoxanthin (from Brown Seaweed), approximately 10 mg; Ganoderma Lucidum, (Ling zhi red Reishi Mushroom), approximately 65 mg, Ginseng (Panex), approximately 5 mg; Glucosamin, approximately 10 mg; Glycine, approximately 3.5 mg; Gold, approximately 1.5 mcg; Grape seed extract, approximately 85 mg; Horse chestnut seed extract, approximately 5 mg; Lactase (from pure plant enzymes), approximately 5 mg; Acidophilus including at least one of Lactobacillus, Bulgarius, and Bifidus, approximately 2.5 mg; L-Arginine, approximately 10 mg; L-Glutahhione, approximately 5 mg; Lipase (from pure plant enzymes), approximately 5 mg; L-Isoleucine, approximately 2.5 mg; L-Leucine, approximately 2.5 mg; L-Lysine, approximately 3.5 mg; L-Valine, approximately 2.5 mg; Maca Root Extract, approximately 5 mg; Magnesium Citrate, approximately 20 mg; Maltase (from pure plant enzymes), approximately 5 mg; Manganese, approximately 10 mcg; Marine Phytoplankton, approximately 5 mg; Methionine, approximately 2.5 mg; Milk Thistle, approximately 15 mg; Morus albus, approximately 25 mg; MSM, approximately 10 mg; Mucuna pruriens, approximately 25 mg; Muira puama, approximately 20 mg; Nettle root extract (Urticaceae urtica), approximately 5 mg; Olive leaf extract, approximately 65 mg; Omega-3 (Alpha-linolenic acid from flaxseed), approximately 7.5 mg; Omega-3 (Docosahexoenoic acid from flaxseed), approximately 7.5 mg; Omega-3 (eicosapentoenoic acid from flaxseed), approximately 7.5 mg; Ornithine, approximately 2.5 mg; Ox Bile, approximately 2.5 mg; Papain (from pure plant enzymes), approximately 7.5 mg; Pfaffia paniculta (Suma), approximately 20 mg; Phosphatidylserine (from soy lecithin), approximately 3.5 mg; Phosphorus, approximately 2.5 mcg; Polygonum Multiflorum, approximately 10 mg; Protease (from pure plant enzymes), approximately 5 mg; Pumpkin Seed Extract, approximately 5 mg; Rhodiola rosea, approximately 3.5 mg; Rhododendron Siberian caucasiuam, approximately 5 mg; Selenium, approximately 5 mcg; Throeonine (herbal), approximately 2.5 mg; Tribulus terrestris, approximately 35 mg; Vitamin A, approximately 30 IU; Vitamin B12, approximately 100 mcg; Vitamin D, approximately 125 IU; and Vitamin E, approximately 35 IU.

2. The composition as recited in claim 1, wherein each of said recited constituents in it respective recited quantity, is selected to maximize efficacy by synergistic interaction thereamoung.

3. The composition as recited in claim 1, wherein each of said recited constituents in it respective recited quantity, is selected for to minimize interactions thereamoung.

4. A composition in liquid form, comprising: water, approximately 20 fluid ounces; Alpha Lipoic Acid, approximately 50 mcg; Amylase, approximately 2.5 mg; Astragalus membranaceus, approximately 2.5 mg; Biotin, approximately 1000 mcg; Calcium, approximately 2.5 mg; Carnosine, approximately 3.5 mg; Cellulase, approximately 2.5 mg; Co-Q10, approximately 5 mg; Cyanotis Vaga, approximately 2.5 mg; Emblica officinalis, approximately 2 mg; Ganoderma Lucidum, (Ling zhi red Reishi Mushroom), approximately 10 mg; Ginkgo Biloba, approximately 1.5 mg; Ginseng (Panex), approximately 7.5 mg; Grape seed extract, approximately 7.5 mg; Guarana seed extract(Paullinia cupana), approximately 7.5 mg; Lactase, (from pure plant enzymes), approximately 2.5 mg; L-Arginine, approximately 1.5 mg; Lipase, from pure plant enzymes), approximately 2.5 mg; Magnesium Chloride, approximately 2.5 mg; Maltase (from pure plant enzymes), approximately 2.5 mg; Milk Thistle, approximately 1 mg; Mucuna pruriens, approximately 1.5 mg; Muira puama, approximately 1.5 mg; Potassium, approximately 10 mcg; Polygonum Multiflorum, approximately 10 mg; Protease, (from pure plant enzymes), approximately 2.5 mg; Rhodiola rosea, approximately 2.0 mg; Selenium, approximately 2 mcg; Tribulus terrestris, approximately 2.0 mg; Vitamin B12, approximately 10 mcg; and Vitamin D, approximately 100 IU.

5. The composition as recited in claim 4, wherein each of said recited constituents in it respective recited quantity, is selected to maximize efficacy by synergistic interaction thereamoung.

6. The composition as recited in claim 4, wherein each of said recited constituents in it respective recited quantity, is selected for to minimize interactions thereamoung.

7. A composition in powder form, comprising: Acidophilis, approximately 2.0 mg; Alfalfa (Medicago sativa), approximately 35 mg; Alpha Lipoic Acid, approximately 7.0 mg; Amylase, from pure plant enzymes), approximately 36 USP Units; Astragalus membranaceus, approximately 36.6 mg; Biotin, approximately 120 mcg; Bismuth, approximately 2.5 mcg; Black Cherry, approximately 2.0 mg; Bromelain (from pure plant enzymes), approximately 1.0 mg; Calcium, approximately 6.7 mg; Cellulase (from pure plant enzymes), approximately 1 mg; Chlorella, approximately 32 mg; Chondroitin, approximately 13 mg; Chromium Picolinate, approximately 2.5 mcg; Copper, approximately 2.0 mcg; Co-Q10, approximately 25 mg; DHEA, approximately 3 mg; Ester-C, approximately 25 mg; Glucosamin, approximately 13 mg; Glutamic Acid HCL, approximately 2.25 mg; Gold, approximately 2.0 mcg; Grape seed extract, approximately 28.9 mg; Hemicellulase, approximately 0.11 mg; Iodine, approximately 2.5 mcg; at least from the group Lactobacillus, Bulgarius, and Bifidus, totaling approximately 1.5 mg; L-Arginine, approximately 45 mg; L-Glutahhione, approximately 25 mg; Lipase (from pure plant enzymes), approximately 28 USP Units; Magnesium Citrate, approximately 6.7 mg; Malt Diastase (from pure plant enzymes), approximately 1.0 mg; Manganese, approximately 2.5 mcg; Molybdenum, approximately 2.5 mcg; Milk Thistle, approximately 68 mg; MSM, approximately 10 mg; Olive leaf extract, approximately 88.9 mg; Omega-3 (Alpha-linolenic acid from flaxseed), approximately 7.5 mg; Omega-3 (Docosahexoenoic acid from flaxseed), approximately 2.5 mg; Omega-3 (eicosapentoenoic acid from flaxseed), approximately 3.7 mg; Omega 6 (Gamma-linolenic Acid), from flaxseed, approximately 2.5 mg; Omega 6 (Linoleic Acid), approximately 3.5 mg; Omega 9 (Oleic Acid), approximately 2.5 mg; Ox Bile, approximately 1.0 mg; Pancreatin, approximately 3.5 mg; Pantothenic Acid, approximately 2.2 mg; Papain (from pure plant enzymes), approximately 1.0 mg; Pepsin, approximately 1.0 mg; Phosphorus, approximately 2.5 mcg; Potassium, approximately 2.5 mcg; Protease (from pure plant enzymes), approximately 36 USP Units; Pumpkin Seed Extract, approximately 5 mg; Selenium, approximately 2.25 mcg; and Vitamin B12, approximately 25 mcg.

8. The composition as recited in claim 7, wherein each of said recited constituents in it respective recited quantity, is selected to maximize efficacy by synergistic interaction thereamoung.

9. The composition as recited in claim 7, wherein each of said recited constituents in it respective recited quantity, is selected for to minimize interactions thereamoung.
